(19) United States
(12) Patent Application Publication
(10) Pub. No.: US 2011/0274666 A1
(43) Pub. Date: Nov. 10, 2011

(54) METHOD OF ENGRAFTING CELLS FROM SOLID TISSUES

(75) Inventors: Rachael TURNER, Carrboro, NC (US); David GERBER, Chapel Hill, NC (US); Oswaldo LOZOYA, Cary, NC (US); Lola M. REID, Chapel Hill, NC (US)

(73) Assignee: University of North Carolina at Chapel Hill

(21) Appl. No.: 13/102,939

(22) Filed: May 6, 2011

Related U.S. Application Data
(60) Provisional application No. 61/332,441, filed on May 7, 2010.

(51) Int. Cl. A61K 35/12; A61M 37/00; A61M 5/31; C12N 5/02

(52) U.S. Cl. 424/93.7; 435/374; 435/325; 604/522; 604/506

(57) ABSTRACT

A method of repairing diseased or dysfunctional organs or of establishing a model system of a disease state is provided. For repairing diseased organs, the method involves engraftment of cells from healthy tissue of the diseased or dysfunctional organ admixed with gel-forming biomaterials and nutrient medium, signaling molecules and extracellular matrix components that can be made insoluble rapidly upon transplantation to form a graft. In this way, the graft mimics the complexity of the native microenvironment with a minimum number of components that allow transplantation of cells to successfully engraft, expand and then rebuild part or the entirety of the diseased or dysfunctional organ. In the case of using grafting methods for establishing a disease model, diseased cells may be transplanted in the biomaterials and into experimental hosts.

### CROSS-REFERENCE TO RELATED PATENT APPLICATIONS

This application claims the benefit of U.S. Provisional Patent Application No. 61/332,441, filed May 7, 2010, which is herein incorporated by reference in its entirety.

### FIELD OF THE INVENTION

The present invention is directed generally to the field of tissue engrafting. More specifically, the invention concerns compositions and methods for the engraftment of cells.

### BACKGROUND OF THE INVENTION

Current methodologies of cell transplant therapies introduce donor cells into hosts via a vascular route, a strategy modeled after hematopoietic therapies. However, hematopoietic cell therapies are relatively easily performed as these cells have evolved to be in suspension and have inherent features that support their homing to specific target tissues. Thus, the many thousands of studies on transplantation of hematopoietic cell subpopulations have little relevance to the transplantation of cells from solid organs, such as skin or internal organs (e.g., liver, lung, heart). Indeed, when cells from solid organs are transplanted via a vascular route, there effects are muted due to inefficient engraftment, poor survival of the cells, and propensity for formation of life-threatening emboli. Hence, the diseases of most solid organs have yet to be treated as successfully as they might be if alternate approaches for transplantation were tried.

The present invention is therefore directed to methods of transplanting cells from solid organs by grafting protocols using available diverse strategies.

### SUMMARY OF THE INVENTION

In one embodiment of the present invention, a method of engrafting tissue of an internal organ in a subject having the internal organ in diseased or dysfunctional condition is provided. The method comprises: (a) obtaining isolated cells of the internal organ from a donor; (b) embedding the cells in biomaterials comprised of extracellular matrix components, optionally admixing a nutrient medium and/or signaling molecules (growth factors, cytokines, hormones), and c) introducing the cells into the target organ, wherein the mixture of cells and biomaterials gels or solidifies in place in the internal organ or on its surface or both in vivo. The internal organs may be liver, biliary tree, pancreas, lung, intestine, thyroid, prostate, breast, uterus, or heart. Suitable signaling molecules are growth factors and cytokines and may include, for example, epidermal growth factor (EGF), hepatocyte growth factor (HGF), stromal cell-derived growth factor (SGF), retinoids (e.g., vitamin A), fibroblast growth factor (FGF, e.g., FGF2, FGF10), vascular endothelial cell growth factor (VEGF), insulin like growth factor I (IGF-I), insulin-like growth factor II (IGF-II), oncostatin M, leukemia inhibitory factor (LIF), transferrin, insulin, glucocorticoids, (e.g., hydrocortisone), growth hormone, any of the pituitary hormones (e.g., follicle stimulating hormone (FSH)), estrogens, androgens, and thyroid hormones (e.g., T3 or T4).

For treatment of a diseased or dysfunctional organs, the donor of cells may be one other than the recipient (allograft) or may also be the subject (autologous) having the internal organ in diseased or dysfunctional condition, provided that the normal cells are obtained from a portion of the internal organ that is not diseased or dysfunctional. For establishing a model system to study a disease, the donor cells can be ones that have the disease and that are transplanted onto/into normal tissue in an experimental host.

The cells may comprise stem cells, mature cells, angioblasts, endothelia, mesenchymal stem cells (from any source), stellate cells, fibroblasts or mixtures of these, In addition, the biomaterials may comprise collagens, adhesion molecules (laminins, fibronectins, nidogen), elastins, proteoglycans, hyaluronans (HAs), glycosaminoglycan chains, chitosan, alginate, and synthetic, biodegradable and biocompatible polymers. Hyaluronans are one of the preferred materials.

The isolated cells of the internal organ may be solidified ex vivo within the biomaterials prior to introducing the cells into the hosts, or in the alternative, injected as a fluid substance and allowed to solidify in vivo. Preferably, the cells are introduced at or near the diseased or dysfunctional tissue, and may be introduced via injection, biodegradable covering, or sponge.

In another embodiment of the present invention, a method of repairing tissue of an internal organ in a subject suffering from the internal organ in a diseased or dysfunctional condition is provided. The method comprises (a) obtaining normal cells of the internal organ from a donor; (b) combining the cells with one or more biomaterials; (c) optionally combining the cell suspension with signaling molecules (growth factors, cytokines), additional cells, or combinations thereof; and (d) introducing the mixture (b) into the subject, wherein the mixture becomes insoluble and forms a graft onto or into the internal organ in vivo.

In yet another embodiment of the present invention, a method of localizing cells of an internal organ onto a surface, into an interior portion, or both of a target internal organ is provided, the method comprising introducing a preparation comprising cells of an internal organ and a solution of one or more hydrogel-forming precursors, in the presence of an effective amount of a cross-linker, onto a surface, into an interior portion, or both of a target internal organ in vivo, which preparation forms a hydrogel comprising cells of an internal organ on a surface, in an interior portion, or both of a target internal organ. The mixture my further comprise nutrient medium, extracellular matrix molecules, and signaling molecules. The solidified mixture, such as a hydrogel, provides a graft into a target internal organ either on its surface, in an interior portion, or both.

The cells may be localized for a period of at least twelve hours, at least twenty-four hours, at least about forty-eight hours, or at least about 72 hours into/onto the target internal organ, which may be liver, pancreas, biliary tree, lung, thyroid, intestine, breast, prostate, uterus, bone, or kidney. In treatment of patients, the donor cells of the internal organ should not be diseased cells (e.g., tumor or cancer cells). However, diseased cells might be considered in a graft when trying to establish an experimental model system of a disease.

The biomaterials that can form hydrogels, or a parallel insoluble complex, can comprise glycosaminoglycans, proteoglycans, collagens, laminins, nidogen, hyaluronans, a thiol-modified sodium hyaluronate, denatured forms thereof (e.g., gelatin), or combinations thereof. A trigger for solidification can be any factor eliciting cross-linking of the matrix components or gelation of those that can gel. The cross-linker may comprise polyethylene glycol diacrylate or a disulfide-containing derivative thereof. Preferably, the insoluble complex of cells and biomaterials possesses a viscosity ranging from about 0.1 to about 100 kPa, preferably about 1 to about 10 kPa, more preferably about 2 to about 4 kPa, and most preferably a stiffness from about 11 to about 3500 Pa.

In still yet another embodiment of the present invention, a method of cryopreserving cells is provided, comprising: (a) obtaining isolated cells; (b) combining the cells with gel-forming biomaterials and, optionally, one or more of isotonic basal medium, signaling molecules (cytokines, growth factors, hormones), and extracellular matrix components (e.g. hyaluronans); and freezing the cell mixture so as to be stored in a −90° C. or −180° C. freezer. The isotonic medium can be CS10 (biolife) or an equivalent isotonic cryopreservation buffer. The signaling molecules can be Suitable signaling molecules are growth factors and cytokines and include, for example, epidermal growth factor (EGF), hepatocyte growth factor (HGF), stromal cell-derived growth factor (SGF), retinoids (e.g., vitamin A), fibroblast growth factor (FGF, e.g., FGF2, FGF10), vascular endothelial cell growth factor (VEGF), insulin like growth factor I (IGF-I), insulin-like growth factor II (IGF-II), oncostatin M, leukemia inhibitory factor (LIF), transferrin, insulin, glucocorticoids, (e.g., hydrocortisone), growth hormone, any of the pituitary hormones (e.g., follicle stimulating hormone (FSH)), estrogens, androgens, and thyroid hormones (e.g., T3 or T4). The extracellular matrix components can be glycosaminoglcyanas, hyaluronans, collagens, adhesion molecules (laminins, fibronectins), proteoglycans, chitosan, alginate, and synthetic, biodegradable and biocompatible polymers, or combinations thereof.

For cryopreservation of the mixtures of cells and biomaterials, mixtures, they may be further combined with a (i) cryoprotectant selected from the group consisting of dimethyl sulfoxide(DMSO), glycerol, ethelyene glycol, ethylenediolethalenediol, 1,2-propanediol, 2,-3 butenediol, formamide, N-methylformamide, 3-methoxy-1,2-propanediol by themselves, and combinations thereof and/or (ii) an additive selected from the group consisting of sugara, glycine, alanine, polyvinylpyrrolidone, pyruvate, an apoptosis inhibitor, calcium, lactobionate, raffinose, dexamethasone, reduced sodium ions, choline, antioxidants, hormones, or combination thereof. The sugar may be trehalose, fructose, glucose, or a combination thereof and the antioxidants may be vitamin E, vitamin A, beta-carotene, or a combination thereof.

### BRIEF DESCRIPTION OF THE FIGURES

FIG. 1 is a schematic of methods according to the invention of grafting cells to various target tissues. These methods include, implantable grafts, injectable grafts, and grafts that can be attached onto the surface of a target organ (“bandaid grafts”).

FIG. 2 provides rheological measurements on hyaluronans prepared with Kubota's Medium (KM-HAs). a) The shear modulus |G*| of KM-HAs, a measurement of mechanical gel stiffness, remains constant while viscoelastic damping |G″/G′|, a measurement of deformation response delay upon external forcing, is negligible within the 0.1 Hz-10 Hz forcing frequency range for each of the formulations tested; error bars: 95% confidence interval of measurements at each frequency tested. b) KM-HAs exhibit shear thinning, i.e. decrease in viscosity with increasing forcing frequencies, across experimental 0.6 l/s−60 l/s shear rate range [0.1 Hz-10 Hz forcing frequency]; upper and lower limits: power law model-based 95% confidence interval (Cox-Merz rule assumption, R2>0.993 for all formulations in the 0.3 l/s-30 l/s shear rate range [0.05 Hz-5 Hz forcing frequency]). Rheological measurements performed only on lettered formulations shown in Table 3.

FIG. 3 shows size, morphology and proliferation data of human hepatic stem cells (hHpSCs) in KM-HAs. Colonies of hHpSCs acquire three-dimensional configurations and exhibit a) spheroid-like agglomeration (bottom left) or folding (middle, top right) upon seeding in KM-HAs [image frame: 900 μm×1200 μm]. Confocal microscopy on histological sections of hHpSC-seeded KM-HAs reveals mixed cell morphology phenotypes after 1 week of culture, with cell sizes of b) about 7 μm, or c) up to 10-15 μm amongst parenchymal cells [cell nuclei in blue from DAPI counterstaining, EpCAM in red for both b) and c), green for either b) CD44, or c) CDH1; image frames b) and c): 150 μm×150 μm; white highlight in b) and c): 15 μm×15 μm]. d) Viability of hHpSCs in KM-HAs, measured by AlamarBlue metabolic reduction, reveals functional recovery and proliferation in KM-HA hydrogels with 1.6% CMHA-S and 0.4% PEGDA (formulation E, Table 3) throughout 1 week of culture; AlamarBlue reduction measurements after 24-hr incubation, normalized with respect to measurements at 2-3 days post-seeding; data reported as mean±standard error.

FIG. 4 provides protein expression of differentiation markers in KM-HA-seeded hHpSCs after 1 week of culture. Colonies of hHpSCs exhibit differential levels of expression for differentiation markers in hHpSCs at the translational level depending on KM-HAs properties. Metabolic secretion rates of human AFP correlate mRNA expression levels across KM-HA formulations. NCAM expression is positive in all KM-HAs, while CD44 expression is richest in KM-HAs with CMHA-S contents of 1.2% or less (lettered formulations A, B, C, D; Table 3). CDH1 expression is positive for KM-HA hydrogels with |G*|<200 Pa and negative for |G*|>200 Pa. Data for human AFP secretion rate reported as mean±standard error. Immunohistochemical staining for EpCAM, NCAM, CD44 and CDH1 performed on 15-20 μm sections (˜2 to 3 hHpSCs thick; hHpSC diameter: 5-7 μm) and imaged by fluorescence microscopy [image frames: 100 μm×100 μm]. KM-HA formulations ordered with respect to increasing stiffness (|G*|=25 Pa for A, |G*|=73 Pa for B, |G*|=140 Pa for E, |G*|=165 Pa for C, |G*|=220 Pa for D, and |G*|=520 Pa for F).

FIG. 5 provides gene expression levels by qRT-PCR for hepatic progenitor markers in KM-HA-grown hHpSCs after 1 week of culture. Comparisons between the mRNA expression levels of markers for hHpSCs and their immediate descendents hHBs (hepatic-specific AFP, EpCAM, NCAM, CD44 and CDH1) show that KM-HA-grown hHpSCs acquire early hHB characteristics at the transcriptional level in passive culture for 1 week. The expression ranges in hHpSCs and freshly isolated hHBs for CD44 are comparable; the expression levels for the remaining markers are statistically distinct, with approximately 2-fold decrease in EpCAM, 3-fold decrease in CDH1, NCAM silencing and AFP enrichment upon hHpSCs differentiation into hHBs. In all KM-HAs, mean expression levels of seeded hHpSCs for AFP, NCAM and CDH1 shifted outside the hHpSC range towards the hHB range, while EpCAM expression is enriched throughout, after 1 week of culture. KM-HA formulations ordered with respect to increasing stiffness (|G*|=25 Pa for A, |G*|=73 Pa for B, |G*|=140 Pa for E, |G*|=165 Pa for C, |G*|=220 Pa for D, and |G*|=520 Pa for F). Expression levels (mean±standard error) were normalized with respect to GAPDH. Measurements in lettered KM-HA formulations (Table 3) compared to hHpSC colonies (green) and freshly isolated hHBs (red) for significance (Student's t-test).

FIG. 6 is a schematic of one embodiment of the disclosed cryopreservation and thawing methods.

FIG. 7 shows the results from in vivo real time imaging of luminescent signal produced by luciferin-producing cells both grafted with hyaluronans versus injected as a cell suspension.

FIG. 8 provides serum human albumin at day 7 post-transplantation in grafted versus cell suspension in both healthy and CCl4 liver injury models.

FIG. 9 shows gene expression of hepatic stem cell phenotype markers. Expression levels are normalized to GAPDH expression, and fold changes are normalized to initial expression in colonies. * denotes p<0.05% significance between experimental condition and initial colony expression. ** denotes p<0.05% significance between experimental condition and initial colony expression as well as significant expression between the two experimental conditions.

FIG. 10 provides data from functional assays of hepatic function over time. A) albumin, B) Transferrin, and C) Urea in three-dimensional hyaluronan culture over time for levels are normalized per cell.

FIG. 11 provides data from mechanical characterization of KM-HAs. a) Stiffness of KM-HAs is controllable and depends on CMHA-S and PEGDA contents. The average shear modulus |G*| increases with increasing CMHA-S and PEGDA contents following a power-law behavior, thus providing direct control of the final mechanical properties of KM-HAs during the initial hydrogel mixing; rheological measurements performed only on lettered formulations shown in Table 3. Error bars±1 standard deviation for measurements in the 0.05 Hz-5 Hz forcing frequency. b) Diffusion in KM-HAs. Measurements of diffusivity within KM-HAs by FRAP (70 kDa fluorescein-labeled dextran) do not differ significantly from Kubota's medium alone; diffusivity measurements performed on all formulations shown in Table 3. Error bars: 95% confidence interval of measurements.

FIG. 12 shows the secretion of human AFP, albumin and urea by hHpSCs seeded into KM-HAs. Colonies of hHpSCs in KM-HAs exhibit some hepatic function with increasing concentrations of human AFP and albumin found in culture media (KM) and equilibration of urea synthesis by day 7 post-seeding. The metabolic secretion rates of human AFP, human albumin and urea are distinctive by day 7 post-seeding amongst KM-HA formulations, with minimum rates for AFP, albumin and decreased urea synthesis in KM-HAs with 1.6% CMHA-S and 0.4% PEGDA (formulation E, Table 3). Left column: metabolite concentration in culture media collected daily after 24-hr incubation for each lettered formulation (Table 3). Right column: metabolite mass secretion rate per hHpSC colony in culture media after 24-hr incubation; total metabolite mass in media is normalized to number of functional hHpSC colonies at each interval as calculated by viability assay with AlamarBlue reduction (approximate number of colonies seeded per sample: 12). All data reported as mean±standard error.

FIG. 13 shows that controlled rate freezing program minimizes liquid-ice phase entropy preventing internal ice damage and allows for repeatable freezing. A) Graph shows chamber temperature in relation to sample temperature (10% DMSO). B) freezing program rates used for Cryomed 1010 system.

FIG. 14 provides both (A) Cell Viability % of cryopreserved fetal hepatic cells post-thaw and (B) Colony counts after 3 weeks of culture for each condition, normalized to fresh samples. Results are reported as mean±standard error of the mean. KM=Kubotas Medium with 10% DMSO and10% FBS. CS10=cryostor, CS10+sup=cryostor10 with KM supplements. 0.05% and 0.10% refer to the HA % supplemented in each sample.

FIG. 15 shows the relative mRNA expression normalized to GAPDH expression. Mean±standard error of the mean. Significance *p>0.05 to Fresh samples. KM=Kubotas Medium with 10% DMSO and 10% FBS. CS10=cryostor, CS10+sup=cryostor10 with KM supplements. 0.05% and 0.10% refer to the HA % supplemented in each sample.

### DETAILED DESCRIPTION OF THE INVENTION

At present, cell transplantation involving cells derived from solid organs is performed typically via a vascular route, and the results routinely provide overwhelming evidence of inefficient engraftment, typically on the order of about 20-30% for mature cells and less than 5% for stem cells. The differential engraftment is due to their sizes which in liver are small for stem cells (typically under 10 μm) and larger for the mature cells (typically >18 μm). Our studies have confirmed this observation. In one study, for example, human hepatic stem cells (hHpSCs) and hepatoblasts (hHBs) were injected into immunocompromised mice by injecting the cells into the spleen. Since the spleen connects directly with the liver, the cells flowed into the liver where they were expected to engraft. However, most of the cells died prior to engraftment or lodged in tissues other than the intended target (ectopic sites).

Even when cells properly did reach their destination, conversion of the cells to fully functional ones was hindered by lack of vascularization, lack of growth (when transplanting mature cells), and highly immunogenic properties of the cells if mature cells were used and necessitating long-term immunosuppression. Other hurdles include the sourcing of clinical grade, high-quality cells and the need to use freshly isolated cells due to difficulties with cryopreservation.

In addition to the inefficiencies and difficulties noted, transplantation of cells from solid organs via a vascular route is dangerous. The cells from solid organs have surface molecules (cell adhesion molecules, tight junction proteins) that make the cells bind to each other rapidly and enhance aggregation. This clumping phenomenon can result in life-threatening pulmonary emboli.

To address some of these hurdles and concerns, the present invention is directed to grafting technologies that involve the delivery of transplanted cells as an aggregate on or in scaffolds that can be localized to the diseased tissue to promote necessary proliferation and engraftment. Thus, the invention takes into account not only the cell type to be transplanted, but also the cell type in combination with the appropriate biomaterials and grafting method for the most efficient and successful transplant therapies. Grafting technologies of the present invention are translatable to therapeutic uses in patients and provide alternative treatments for regenerative medicine to reconstitute diseased or dysfunctional tissue.

Cell Sourcing

According to the invention, desired cell populations may be obtained directed from a donor having “normal,” “healthy” tissue and/or cells, meaning any tissue and/or cells that is/are not afflicted with disease or dysfunction. Of course, such a cell population may be obtained from a person suffering from an organ with disease or dysfunction, albeit from a portion of the organ that is not in such a condition. The cells may be sourced from any appropriate mammalian tissue, regardless of age, including fetal, neonatal, pediatric, and adult tissue. If experimental models of a disease state are to be established, then one can utilized diseased cells in the grafts that are to be transplanted into an appropriate experimental host.

More specifically, cells may be sourced for different therapies from “lineage-staged” populations based on the therapeutic need. For example, later-stage “mature” cells may be preferred in cases where there is a need for rapid acquisition of functions offered only by the late lineage cells, or if the recipient has a lineage-dependent virus that preferentially infects the stem cells and/or progenitors such as occurs with hepatitis C or papilloma virus. In any event, “progenitor” cells may be used to establish any of the lineage stages of their respective tissue(s).

For a discussion of lineage-staged liver cell populations and method of their isolation, see U.S. patent application Ser. Nos. 11/560,049 and 12/213,100, the disclosures of which are both incorporated in their entirety herein by reference. Briefly, there are at least eight maturational lineage stages that are intrahepatic. Below are given those stages and brief statements about them:

Lineage Stage 1: Human hepatic stem cells (hHpSCs) are multipotent cells, located within the ductal plates of fetal and neonatal livers and the canals of Hering in pediatric and adult livers. These cells usually range from 7-10 μm in diameter and have high nucleus to cytoplasmic ratios. They are tolerant of ischemia, can be found in cadaveric livers for more than 48 hours after systolic death, and form colonies of hHpSCs capable of differentiation to mature cells. These cells constitute approximately 0.5-2% of the parenchyma of livers of all age donors.

Lineage Stage 2: The hepatoblasts (hHBs) are the immediate descendents of the hHpSCs and are the liver's probable transit amplifying cells. They are located just outside the stem cell niche proper. These cells are larger (10-12 μm) with higher amounts of cytoplasm and are found in vivo throughout the parenchyma in fetal and neonatal livers and near the ends of or adjacent to the canals of Hering in pediatric and adult livers. With age, the hepatoblasts decline in numbers to <0.01% of the parenchymal cells in postnatal livers. This population of cells has been shown to expand during regenerative processes especially those associated with certain diseases such as cirrhosis. Hepatoblasts mature into either hepatocytes (H) or cholangiocytes, also called biliary epithelia (B):

Lineage Stage 3H and 3B: Committed (unipotent) hepatocytic (3H) and cholangiocyte progenitors—biliary progenitors (3B) are found within the liver. These unipotent precursors give rise to only one adult cell type, and no longer express some of the stem cell genes (e.g., low or no levels of expression for CD133/1, Hedgehog proteins (Sonic/Indian) but express genes typical for cells in the fetal tissues.

Lineage Stage 4 H and 4B: Periportal adult parenchymal cells comprise relatively small hepatocytes (4H) and intrahepatic biliary epithelia (4B). The hepatocytes are diploid, are approximately 18 μm in diameter, and express multiple factors/enzymes associated with gluconeogenesis such as PEPCK, connexins 26 and 32.

The cholangiocytes of this stage (4B) are diploid, are approximately 6-7 μm in diameter, line a portion of the canals of Hering, and express various genes including aquaporins 1 and 4, MDR1, secretin receptor, but not CL⁻/HC03⁻ exchanger or somatostatin receptor.

Lineage Stage 5 H and 5B: Cells of this stage comprise relatively larger hepatocytes (5H) and cholangiocytes (5B), both diploid. The size of the hepatocytes is approximately 22-25 μm in diameter, and they are found in the midacinar zone. The midacinar hepatocytes express high levels of albumin and tyrosine aminotransferase (TAT); especially characteristic is that they express transferrin as a protein (by contrast, lineage stages 1-4 express it only as mRNA.

Lineage stage 5B cholangiocytes are approximately 14 μm in diameter, located within the intralobular ducts, and express CFTR, Secretin receptor, somatostatin receptor, MDR1 and MDR3, and the CL⁻/HC03⁻ exchanger.

Lineage Stage 6H: The diploid pericentral hepatocytes of stage 6 can form colonies in culture, but have limited capacity to expand and essentially no capacity to be subcultured. The percentage of these declines with age (in parallel with an increase in the percentage of tetraploid pericentral cells). In addition to albumin, TAT, and transferrin, they express also strongly a number of the P450s such as P450-3A, glutamine synthetase (GT), heparin proteoglycans, and the genes associated with urea formation.

Lineage Stage 7H: This stage comprisese tetraploid pericentral parenchymal cells that are no longer able to undergo complete cell division. They can undergo DNA synthesis but with limited capacity for cytokinesis. They are much larger cells (>30 m in diameter) and express high levels of the genes that become apparent in lineage stages 5-6.

Lineage Stage 8: Apoptotic Cells: express various markers of apoptosis and demonstrate DNA fragmentation.

In addition to the cells required to provide the “functions” per se of a diseased or dysfunctional internal organ, the graft preferably includes additional cellular components that preferably mimic the categories of cells comprising the epithelial-mesenchymal cell relationship, the cellular foundation of all tissues. Epithelial-mesenchymal cell relationships are distinct at every maturational lineage stage. Epithelial stem cells are partnered with mesenchymal stem cells and their maturation is coordinate with each other as they mature to all the various adult cell types within a tissue. The interactions between the two are mediated by paracrine signals that comprise soluble signals (e.g., growth factors) and extracellular matrix components.

In livers, for example, the hepatic stem cells (HpSCs) give rise to hepatocytes and to cholangiocytes. The mesenchymal partners for the HpSCs are angioblasts. There is evidence to indicate that angioblasts give rise to both endothelial cell precursors and to hepatic stellate cell precursors, the mesenchymal cell partners for intrahepatic lineage stage 2 parenchyma, the hepatoblasts (HBs). The endothelial cell precursors mature in subsequent lineage stages to be endothelia that become the mesenchymal partners for the lineage stages of hepatocytes. The stellate cell precursors cells give rise to stellate cells, and then to stromal cells, and then to myofibroblasts, the mesenchymal cellspartners for cholangiocytes.

The formation of the liver, called hepatogenesis, is regulated through signals from the angioblasts in the embryonic mesenchyme associated with the heart. During the initial stages of liver development, fibroblast growth factors (FGFs) are secreted from pre-cardiac mesoderm while bone morphogenetic proteins (BMPs) are delivered from the mesenchyme. These newly specified hepatic cells then break away and migrate into the surrounding mesenchyme and interact with precursors to both endothelia and stroma. The mesenchymal cells remain in contact with hepatic cells throughout development.

Human hepatic stem cells (hHpSCs) require contact with mesenchymal cells for survival. They will self-replicate, that is remain as hHpSCs when on feeders of angioblasts. They lineage restrict to hepatoblasts, if cultured on feeders of hepatic stellate cells. They mature into adult hepatocytes if cultured on mature endothelia and to cholangiocytes if on mature stroma (e.g. mature stellate cells or myofibroblasts). The control of the fate of the stem cells by the feeders has been shown to be due to the exact combinations of paracrine signals produced in each of the epithelial-mesenchymal relationships in the lineages.

According to one embodiment of the invention, the isolated cell populations are combined with known paracrine signals (discussed below) and “native” epithelial-mesenchymal partners, as needed, to optimize the graft. Thus, the grafts will comprise the epithelial stem cells, the hepatic stem cells, mixed together with their native mesenchymal partners, angioblasts. For a transit amplifying cell niche graft, hepatoblasts can be partnered with hepatic stellate cells and endothelial cell precusors. In some grafts one can make a mix of the two sets: hepatic stem cells, hepatoblasts, angioblasts, endothelial cell precursors, hepatic stellate cell precursors cells to optimize the establishment of the liver cells in the host tissue. The microenvironment of the graft into which the cells are seeded will be comprised of the paracrine signals, matrix and soluble signals, that are produced at the relevant lineage stages used for the graft.

Grafts can also be tailored to manage a disease state. For example, to minimize effects of lineage dependent viruses (e.g., certain hepatitis viruses) that infect early lineage stages and then mature coordinately with the host cells, one can prepare grafts of later lineage stage (e.g., hepatocytes and their native partners, sinusoidal endothelial cells) that are non-permissive for viral infection. Grafts can be used also to establish a disease model by using diseased cells in a graft that is transplanted into/onto a target organ in an experimental animal model.

An example of a stem cell graft, using liver cell therapies as a model, would comprise the hepatic stem cells, angioblasts and hepatic stellate cell precursors. In contrast, a graft of “mature” liver cells would comprise hepatocytes, mature endothelial cells and pericytes, which are the mature stellate cells. For a discussion of the epithelial-mesenchymal cell relationship of livers, see U.S. patent application Ser. No. 11/753,326, the disclosure of which is incorporated in its entirety herein by reference.

The issue of vascularization is important for all grafts, and therefore should be implanted in location conducive to vascularization (e.g., liver). For most disease conditions, stem cell grafts are preferred, given their expansion potential, their ability to mature into all of the adult cell types, their tolerance for ischemia, enabling their sourcing from cadaveric tissue, and their minimal, if any, immunogenicity.

Grafting Materials

The use of gel-forming biomaterials according to the invention provides a scaffold for cell support and signals that assist in the success of the grafting and regenerative processes. As tissue of solid organs in an organism undergo constant remodeling, dissociated cells tend to reform their native structures under appropriate environmental conditions. The cells may be combined with one or more of a nutrient medium (e.g., RPM 1640), signaling molecules (e.g., insulin, transferrin, VEGF) and one or more extracellular matrix components (e.g., hyaluronans, collagens, nidogen, proteoglycans).

In all tissues, the paracrine signaling comprises both soluble (myriad growth factors and hormones) and insoluble (extracellular matrix (ECM) signals). Synergistic effects between the soluble and (insoluble) matrix factors can dictate growth and differentiative responses by the transplanted cells. The matrix components are the primary determinants of attachment, survival, cell shape (as well as the organization of the cytoskeleton), and stabilization of requisite cell surface receptors that prime the cells for responses to specific extracellular signals.

The ECM is known to regulate cell morphology, growth and cellular gene expression. Tissue-specific chemistries similar to that in vivo may be achieved ex vivo by using purified ECM components. Many of these are available commercially and are conducive to cell behavior mimicking that in vivo.

Suitable matrix components include collagens, adhesion molecules (e.g., cell adhesion molecules (CAMs), tight junctions (cadherins), basal adhesion molecules (laminins, fibronectins), gap junction proteins (connexins), elastins, and sulfated carbohydrates that form proteoglycans (PGs) and glycosaminoglycans (GAGs). Each of these categories defines a genus of molecules. For example, there are at least 25 collagen types present, each one encoded by distinct genes and with unique regulation and functions. Additional biomaterials include inorganic, natural materials like chitosan and alginate as well as many synthetic, biodegradable and biocompatible polymers. These materials are often “solidified” (e.g., made into a gel or an insoluble material) through methods including thermal gelation, photo cross-linking, or chemical cross-linking or exposure to microenvironment (e.g., high salt) that elicit insolubility of the materials. With each method, however, it is necessary to account for cell damage (e.g., from excessive temperature ranges, UV exposure). For a more detailed discussion of biomaterials, specifically the use of hyaluronan hydrogels, see U.S. patent application Ser. No. 12/073,420, the disclosure of which is incorporated in its entirety herein by reference.

The particular selection of which matrix components may be guided by gradients in vivo, for example, that transition from components found in association with the stem cell compartment to that found in association with the late lineage stage cells. The graft biomaterials preferably mimic the matrix chemistry of the particular lineage stages desired for the graft. The efficacy of the chosen mix of matrix components may be assayed in ex vivo studies using purified matrix components and soluble signals, many of which are available commercially, according to good manufacturing practice (GMP) protocol. The biomaterials selected for the graft preferably elicit the appropriate growth and differentiation responses required by the cells for a successful transplantation.

Concerning the liver organ, the matrix chemistry associated with liver parenchymal cells, and outside of the stem cell and transit amplifying cell niches, is present in the Space of Disse, the area located between the parenchyma and the endothelia or other forms of mesenchymal cells. In addition to a change in cell maturity within the different zones of the liver, a change in matrix chemistries is also observed. The matrix chemistry periportally in zone 1 is similar to that found in fetal livers and consists of type III and type IV collagens, hyaluronans (HA), laminins, and forms of chondroitin sulfate proteoglycans. This zone transitions to a different matrix chemistry in the pericentral zone 3, containing type I collagen, fibronectin, and unique forms of heparin and heparan sulfate proteoglycans.

The stem cell niche of the liver has been characterized partially and found to comprise hyaluronans, laminin forms (e.g., laminin 5) that bind to alpha 6-beta 4 integrin, type III collagen and unique forms of minimally sulfated chondroitin sulfate proteoglycans (CS-PGs) There are limited amounts of type IV collagen and no type I collagen in this niche.

This niche matrix chemistry transitions to that associated with the transit amplifying cell compartment and is comprised of type IV collagen, forms of laminin that bind to other integrins (αβ1), and forms of GAGs and PGs that include forms of CS-PGs with higher sulfation, dermatan sulfate-PGs, and to specific forms of heparan sulfate-PGs (HS-PGS).

The transit amplifying cell compartment transitions to yet later lineage stages, and with each successive stage, the matrix chemistry becomes more stable (e.g., more highly stable collagens), turns over less, and contains more highly sulfated forms of GAGs and PGs. The most mature cells are associated with forms of heparin-PGs (HP-PGs), meaning that myriad proteins (e.g., growth factors and hormones, coagulation proteins, various enzymes) can bind to the matrix and be held stably there via binding to the discrete and specific sulfation patterns in the GAGs. Thus, the matrix chemistry transitions from its start point in the stem cell niche having labile matrix chemistry associated with high turnover and minimal sulfation (and therefore minimal binding of signals in a stable fashion near to the cells) to stable matrix chemistries with increasing amounts of sulfation (and therefore higher and higher levels of signal binding and held near to the cells).

Hence, the present invention takes into consideration that the chemistry of the matrix molecules changes with maturational stages, with host age, and with disease states. Grafting with the appropriate materials should optimize engraftment of transplanted cells in a tissue, prevent dispersal of the cells to ectopic sites, minimize embolization problems, and enhance the ability of the cells to integrate within the tissue as rapidly as possible. Moreover, the factors within the graft can also be chosen to minimize immunogenicity problems.

In the case of human livers, cells may be cultured under serum free conditions. Human hepatic stem cell or hepatoblasts (hHpSC or hHB) can be grafted by themselves, or in combination with angioblasts/endothelial cell precursors and stellate cell precursors cells. Cells can be suspended in thiolated and chemically-modified HA (CMHA-S, or Glycosil, Glycosan BioSystems, Salt Lake City, Utah) containing medium (HA-M) and in KM (Kubota's Medium) and loaded into one of the syringes of a set of paired syringes. The other syringe may be loaded with a cross-linker, e.g., poly(ethylene glycol) diacrylate or PEGDA, prepared in KM (or with the conditions required to elicit insolubility of the biomaterials). The two syringes are coupled by a needle that flares into two luer lock connections. Thus, the cells in hydrogel and the cross-linker can emerge through one needle to allow for rapid cross-linking of the CMHA-S into a gel upon injection (or insolubility of the biomaterials by alternate means).

The cell suspension in CMHA-S and crosslinker can be either directly injected or grafted to the liver using the omentum tissue to form a pouch. Alternatively, the cells may be encapsulated in Glycosil without the use of a PEGDA crosslinker by allowing the suspension to stand overnight in air, leading to disulfide bond crosslinking to a soft, viscous hydrogel. In addition, other thiol-modified macromonomers, e.g., gelatin-DTPH, heparin-DTPH, chondroitin sulfate-DTPH, may be added to give a covalent network mimicking the matrix chemistry of particular niches in vivo. In another manifestation, polypeptides containing cysteine or thiol residues can be coupled to the PEGDA prior to adding the PEGDA to the Glycosil, allowing specific polypeptide signals to be incorporated into the hydrogel. Alternatively, any polypeptide, growth factor or matrix component such as an isoform of a collagen, laminin, vitronectin, fibronectin, etc., may be added to the Glycosil and cell solution prior to crosslinking, allowing passive capture of important polypeptide components in the hydrogel.

Hyaluronans: Hyaluronans (HAs) are members of one of the 6 large glycosaminoglycan (GAG) families of carbohydrates, all being polymers of a uronic acid and an aminosugar [1-3]. The other families comprise the chondroitin sulfates (CS, [glucuronic acid-galactosamine]_{X}), dermatan sulfates (DS, more highly sulfated [glucuronic acid-galactosamine]_{X}), heparan sulfates (HS, [glucuronic acid-glucosamine]_{X}), heparins (HP, more highly sulfated [gluronic acid-glucosamine]_{X}) and keratan sulfates (KS, [galactose-N-acetylglucosamine]_{X}).

HAs are composed of a disaccharide unit of glucosamine and gluronic acid linked by β1-4, β1-3 bonds. Biologically, the polymeric glycan is composed of linear repeats of a few hundreds to as many as 20,000 or more of disaccharide units. The HAs have molecular masses typically ranging from 100,000 Da in serum to as much as 2,000,000 in synovial fluid, to as much as 8,000,000 in umbilical cords and the vitreous. Because of its high negative charge density, HA attracts positive ions, drawing in water. This hydration allows HA to support very compressive loads. HAs are located in all tissues and body fluids, and most abundant in soft connective tissue, and the natural water carrying capacity lends itself to speculation to other roles including influences of tissue form and function. It is found in extracellular matrix, on the cell surface and inside the cell.

Native forms of HA chemistry are diverse. The most common variable is the chain length. Some are high molecular weight due to having long carbohydrate chains (e.g. those in the coxcomb of gallinaceous birds and in umbilical cords) and others are low molecular weight due to having short chains (e.g. from bacterial cultures). The chain length of HAs plays a key role in the biological functions elicited. A low molecular weight HA (below 3.5×10⁴ kDa) may induce the cytokine activity that is associated with matrix turnover and is shown to be related to inflammation in tissues. A high molecular weight (above 2×10⁵ kDa) may inhibit cell proliferation. Small HA fragments, between 1-4 kDa, have been shown to increase angiogenesis.

Native forms of HA have been modified to introduce desired properties (e.g., modification of the HAs to have thiol groups allowing the thiol to be used for binding of other matrix components or hormones or for novel forms of cross-linking). Also, there are forms of cross-linking that occur in nature (e.g., regulated by oxygen) and yet others that have been introduced artificially by treatment of native and modified HAs with certain reagents (e.g., alkylating agents) or, as noted above, establishment of modified HAs that make them permissive to unique forms of cross-linking (e.g., disulfide bridge formation in the thiol-modified HAs).

According to the invention, thiol-modified HAs and in situ polymerizable techniques used for them are preferred. These techniques involve disulfide crosslinking of thiolated carboxymethylated HA, known as CMHA-S or Glycosil. For in vivo studies, HA with lower molecular weight, e.g., 70-250 kDa, can be used, since the crosslinking, either disulfide or PEGDA, creates a hydrogel of very high molecular size. A thiol-reactive linker, polyethylene glycol diacrylate (PEGDA) crosslinker, is suitable for both cell encapsulation and in vivo injections. This combined Glycosil-PEGDA material crosslinks through a covalent reaction and in a matter of minutes, is biocompatible and allows for cell growth and proliferation.

The hydrogel material, Glycosil, takes into account the gel properties conducive to tissue engineering of stem cells in vivo. Glycosil is part of the semi-synthetic extracellular matrix (sECM) technology available from Glycosan Biosciences in Salt Lake City, Utah. A variety of products in the Extracel and HyStem trademarked lines are commercially available. These materials are biocompatible, biodegradable, and non-immunogenic.

Furthermore, Glycosil and Extralink can be easily combined with other ECM materials for tissue engineering applications. HA can be obtained from many commercial sources, with a preference for bacterial fermentation using either Streptomyces strains (e.g., Genzyme, LifeCore, NovaMatrix, and others) or bacterial-fermentation process using Bacillus subtilis as the host in an ISO 9001:2000 process (unique to Novozymes).

The ideal ratios of the cell populations should replicate those found in vivo and in cell suspensions of the tissue. A mix of cells allows for maturation of progenitor cells and/or maintenance of the adult cell types concomitant with the development of requisite vascularization. In this way, a composite microenvironment using hyaluronans as a base for a complex containing multiple matrix components and soluble signaling factors and designed to mimic specific microenvironmental niches comprised of specific sets of paracrine signals produced by an epithelial cell and a mesenchymal cell at a specific maturational lineage stage is achieved. The following are examples:

TABLE 1
REPRESENTATIVE NICHE GRAFTS FOR PROGENITORS

| | | TRANSIT |
|---|---|---|
| | STEM CELL | AMPLIFYING CELL |
| | NICHE GRAFT | NICHE GRAFT |
| Cellular | hHpSCs, angioblasts | Hepatoblasts, hepatic |
| components | | stellate cell precursors, |
| | | endothelial cell |
| | | precursors(or |
| | | mesenchymal stem cell) |
| Base | Kubota's Medium | Kubota's Medium or a |
| Medium | (optimal for endodermal | medium optimal for the |
| | progenitors) or a medium | specific endodermal |
| | tailored for the specific | progenitors tailored for the |
| | category of stem cells | transit amplifying cells |
| Additional | LIF, VEGF | EGF, HGF, VEGF |
| soluble | | |
| factors | | |
| [tailored | | |
| for liver] | | |
| Base | HA or chemically- | HA or chemically- |
| Scaffold | modified HA as an sECM | modified HA as an sECM |
| Other | Type III collagen, | Type IV collagen, any of a |
| Matrix | embryonic forms of laminin | number of forms of |
| Components | (e.g. laminin 5 that binds to | laminins, CS-PGs, HS- |
| | a6/β4 integrin); chondroitin | PGs |
| | sulfate-PG (novel form | |
| | found in niches) | |

TABLE 2
REPRESENTATIVE NICHE GRAFTS FOR
MATURE PARENCHYMAL CELLS

| | | Lineage stage 5, |
|---|---|---|
| | Lineage stage | Intrahepatic |
| | 4 Hepatocytes | Cholangiocyte |
| Cellular | Periportal Hepatocytes | Intrahepatic cholangiocyte |
| components | (~18 μm in diameter); | (~15 μm in diameter); |
| | periportal endothelial cells | stromal cells |
| Base | Kubota's Medium (or a | Kubota's Medium (or one |
| Medium | medium tailored for adult | tailored for adult |
| | hepatocytes): addition of | cholangiocytes): addition |
| | copper (10E-10M), | of copper (10E-10M), |
| | calcium (0.6 mM), EGF | calcium (0.6 mM), EGF |
| | (10 ng/ml) | (10 ng/ml), |
| Additional | VEGF (10 ng/ml), EGF | PDGF (10 ng/ml), EGF |
| soluble | (10 ng/ml), T3 (10E-9M), | (10 ng/ml), HGF (10 |
| factors | Glucocorticoids (10E-8M) | ng/ml), T3 (10E-9M), |
| [tailored | | Glucocorticoids (10E-8M) |
| for liver] | | |
| Base | HA or chemically- | HA or chemically- |
| Scaffold | modified HA as an ECM | modified HA as an ECM |
| Other Matrix | Type III collagen, | Type IV collagen, any of a |
| Components | embryonic form of laminin | number of forms of |
| | (e.g. laminin 5 that binds to | laminin that bind to α/β1, |
| | α6/β4 integrin); chondroitin | CS-PGs, HS-PGs |
| | sulfate-PG (novel form | |
| | found in niches) | |

The microenvironment of a stem cell niche in the liver consists of the paracrine signals between the hepatic stem cell and angioblasts. It is comprised of hyaluronans, type III collagen, specific forms of laminin (e.g., laminin 5), a unique form of chondroitin sulfate proteoglycan (CS-PG) that has almost no sulfation and a soluble signal/medium composition close to or exactly that of “Kubota's Medium”, a medium developed for hepatic progenitors. No other factors are strictly required, though effects can be observed by supplementation with stem cell factor, leukemia inhibitory factor (LIF), and/or certain interleukins (e.g., IL 6, IL 11 and TGF-β1). The stem cell niche form of CS-PG is not yet available

The transit amplifying cell microenvironment in the liver is morphologically between that of the hepatoblasts and hepatic stellate cells. The components of this microenvironment include hyaluronans, type IV collagen, specific forms of laminins that bind to β1 integrin, more sulfated CS-PGs, forms of heparan sulfate-proteoglycans (HS-PGs), and soluble signals that include Kubota's Medium supplemented further with epidermal growth factor (EGF), hepatocyte growth factor (HGF), stromal cell-derived growth factor (SGF), and retinoids (e.g., vitamin A).

Grafting Methods

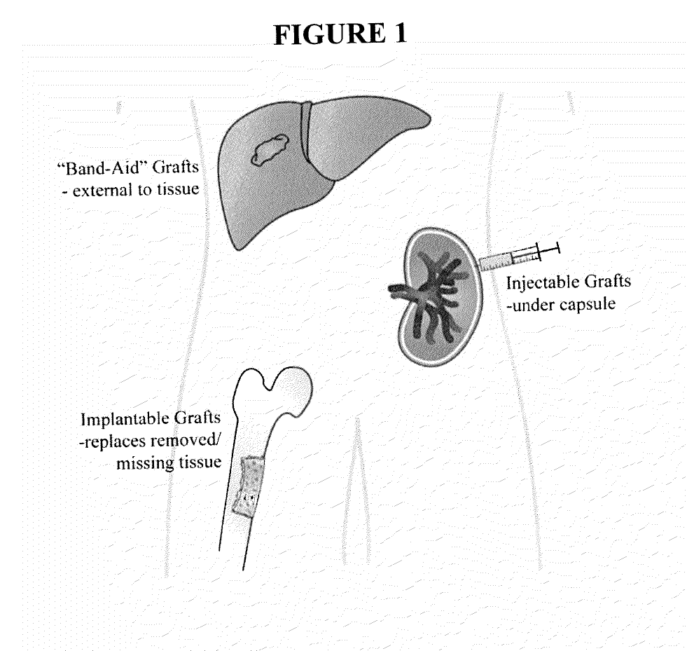

Depending on the tissue type, an appropriate grafting method may be selected. For tissues where grafts would replace a diseased or missing tissue (bone, for example), an implantable graft is suitable. Then, depending on the chosen method, appropriate biomaterials may be chosen to compliment the method. Different methods will be required. For example, in the bone example, a solid matrix allows cells to be seeded with necessary growth factors into the matrix, cultured, and then implanted into the patient. FIG. 1.

Injectable grafts have an advantage in that they can fill any deficit shape or space (e.g., damaged organs or tissues). According to this method, cells are co-cultured and injected in a cell suspension embedded in gelable biomaterials, which solidifies in situ using various crosslinking methods. The mixture may be directly injected into the host tissue or organ (e.g., liver); injected under the organ capsule, any membrane enveloping an organ or tissue; injected into a pouch formed by folding over a part of the omentum onto itself and gluing it to form a pouch; or forming a pouch by using surgical glue to affix another material (e.g. spider silk) to the surface of the organ and injecting the mixture into it.

Direct injection can consist of injection under a liver's Glisson capsule and into the parenchyma at multiple sites, but as few as possible to avoid hydrostatic pressure from the hydrogel that may cause damage to the liver tissue. Injection of the hepatic stem cell niche grafts into the livers is done using a double barreled syringe as described hereinabove. Briefly, the cells-matrix-medium mixture is loaded into one side of the syringe with connecting needle to the other syringe containing the cross-linker PEGDA. The mixture can be injected through a 25 gauge needle directly into the livers and instantly cross-linked to form a hydrogel. The use of CMHA-S with PEGDA at pH 7.4 allows cell encapsulation as well as injection in vivo, since the crosslinking reaction occurs within a few minutes or up to 10-20 min time frame depending on the concentration of the cross-linker.

Inorganic, natural materials like chitosan, alginate, hylauronic acid, fibrin, gelatin, as well as many synthetic polymers can suffice as biomaterials for injections. These materials are often solidified through methods including thermal gelation, photo cross-linking, or chemical cross-linking. The cell suspension may also be supplemented with soluble signals or specific matrix components. Since these grafts can be relatively easily injected into a target area, there is no (or minimal) need for invasive surgery, which reduces cost, patient discomfort, risk of infection, and scar formation. CMHA may also be used for injectable material for tissue engineering due to its long-lasting effect while maintaining biocompatibility. Cross-linking methods also maintain the material biocompatibility, and its presence in extensive areas of regenerative or stem/progenitor niches make it an attractive injectable material.

In some embodiments, a graft may be designed for placement onto the surface of an organ or tissue, in which case the graft would be held in place with a biocompatible and biodegradable covering (“band aid”). For some abdominal organs, this covering could be from autologous tissues. For example, grafting of liver cells (e.g., hepatic progenitors) onto the surface of livers can by done by using the host omentum to form an injection pouch. The omentum is lifted from its location within the abdominal cavity and glued onto the liver using surgical glue (e.g., fibrin glue, dermabond) to form a pouch for the transplant material. The double barreled syringe can again be used to inject the matrix material within the pouch on the exterior of the liver.

As well, a graft may be formed within the omentum pouch, independent of the target tissue. For example, instead of grafting a transplant into or onto the target tissue, one can use the grafting method for ectopic sites. The graft could be established within an omentum pouch, which pouch would be formed by fibrin glue (or equivalent). This approach may be especially suitable for liver grafts when the host liver is too scarred or has some other parameter that would block success of a graft into the tissue itself. Another example is of endocrine cells (e.g., islets) that have a primary requirement to be able to access the vascular supply. A graft of endocrine cells such as islets could be made into an omentum pouch.

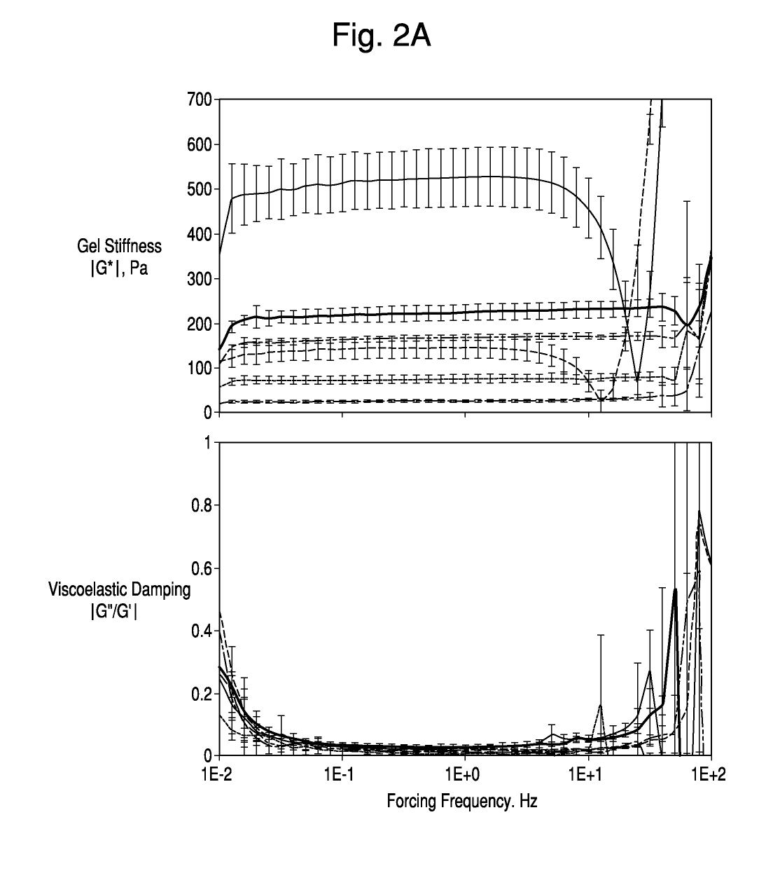

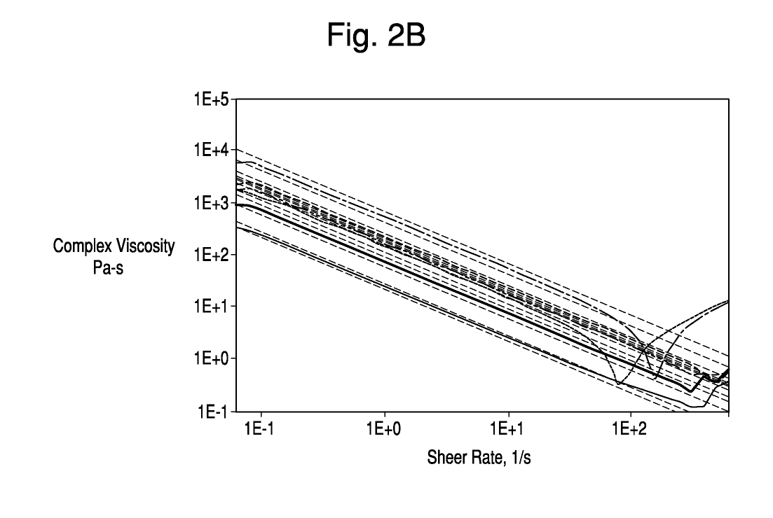

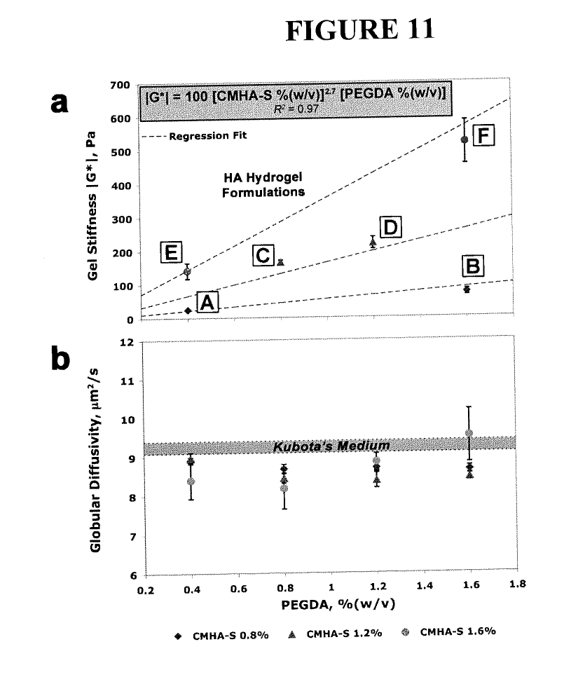

The present inventors have learned that the stiffness, viscoelastic properties and viscosity of KM-HA hydrogels can depend on CMHA-S and PEGDA content. KM-HA hydrogels, for example, maintain a constant stiffness across a broad forcing frequency range while exhibiting perfectly elastic behavior (FIG. 2a) and shear thinning, in which their viscosity decreases with increasing forcing frequency (FIG. 2b). These KM-HA hydrogels can yield shear moduli ranging from 11 to 3500 Pa with different PEGDA and CMHA-S concentrations when mixed in buffered distilled water, but these values can be modulated by using diverse basal medium like Kubota's medium (FIG. 2 and FIG. 11).

The mechanical properties of the ECM into which the cells to be transplanted are seeded can have profound effects on signaling, transport, and on the ability of the cells to respond to mechanical forces using mechanisms collectively known as mechanotransduction. For example, human hepatic progenitors, such a hepatic stem cells, can differentiate when seeded in mechanically rigid grafts, such as within stiff HA hydrogels having a yield shear moduli ranging from 11 to 3500 Pa with different PEGDA and CMHA-S concentrations when mixed in buffered distilled water (FIG. 2).

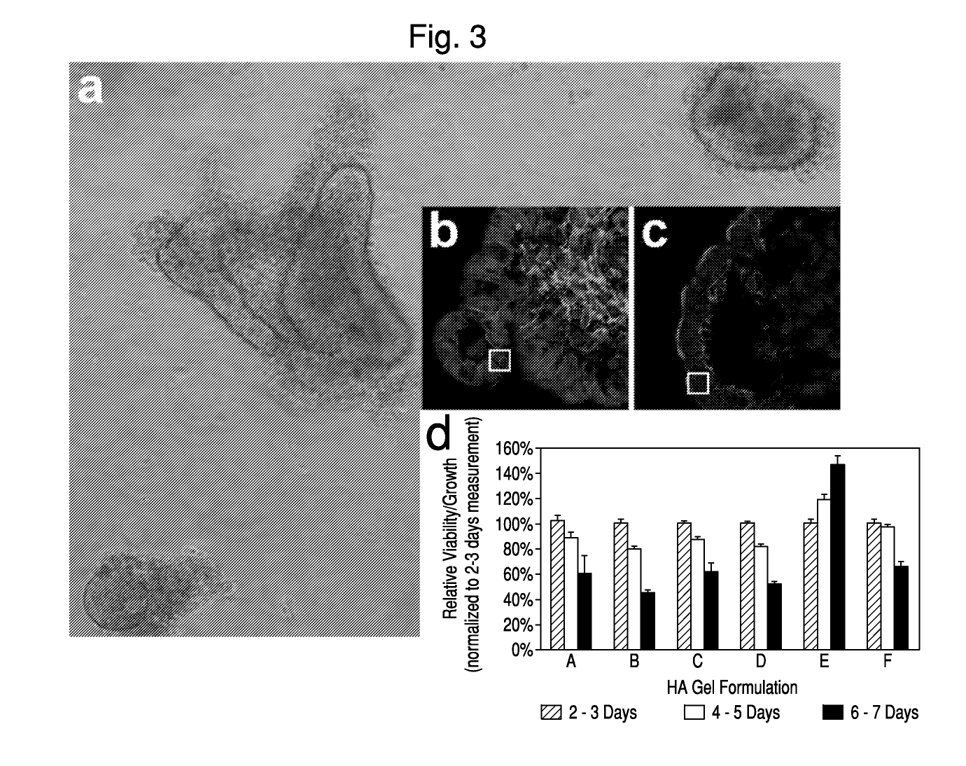

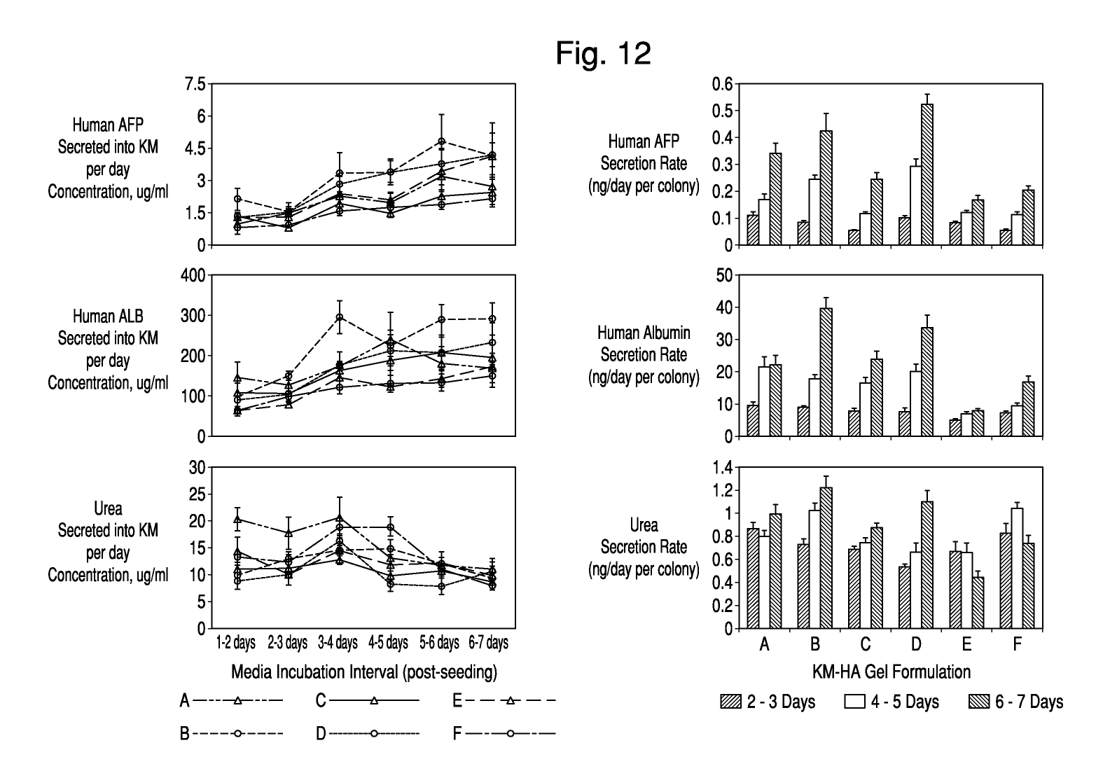

Hepatic stem cell colonies have distinct metabolic activities in accordance with the composition of KM-HA hydrogel hosting them. Absolute secretion is comparable across KM-HA formulations for indicators of hepatic function (AFP, albumin and urea) throughout culture; however, absolute secretion coupled with metabolic efficiency depicts a selection process that depends on the HA content. (FIG. 12). In this process, secretion rates increase under metabolic duress for KM-HA hydrogels with CMHA-S contents lower than 1.2%; in contrast, secretion rates are comparatively poor in KM-HA hydrogels with more CMHA-S (1.6%) and higher metabolic function—or even increased viability, as in formulation E (FIG. 3d). Because hHpSCs and hHBs exhibit different metabolic capabilities, KM-HA hydrogels can select for expansion or differentiation of hepatic progenitors.

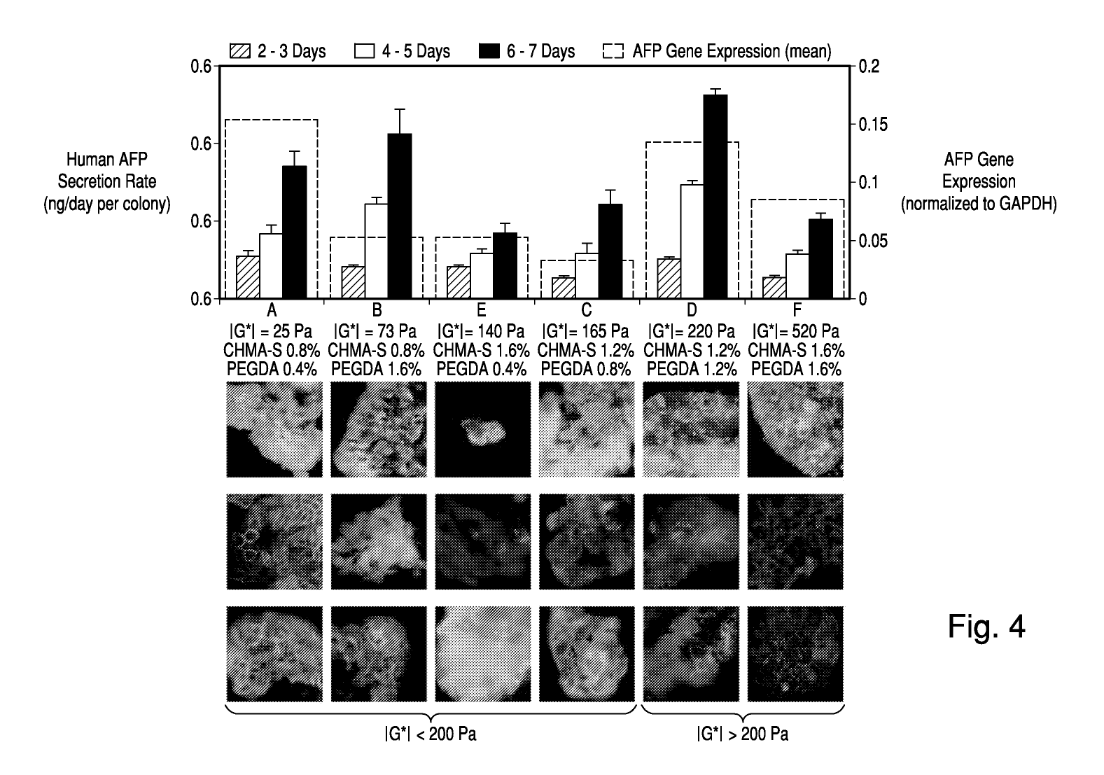

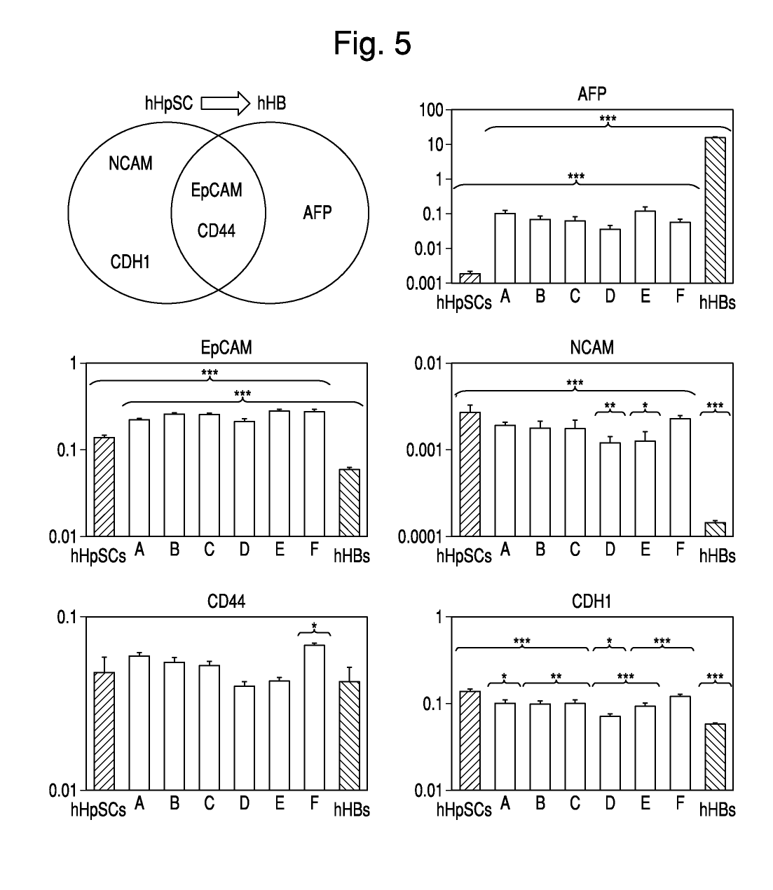

Expression analysis of differentiation markers in hepatic progenitors confirms that differentiation takes place within KM-HA hydrogels, as evidenced by an increased overall gene expression of EpCAM beyond established levels for hHpSC colonies on plastic plates (FIG. 5), as well as heterogeneous NCAM expression across colonies towards the outer boundaries and on the apical surface of external cells. (FIG. 4). CD44 was found expressed on both hHpSCs and hHBs at the mRNA expression level. (FIG. 5). Unlike NCAM, greater CD44 expression was observed in KM-HA hydrogels over CMHA-S contents of 1.2% or less (FIG. 4).

mRNA expression levels depend on the stiffness of KM-HA hydrogels (FIG. 5), that this dependency on stiffness defines two regimes (one at low graft rigidities where gene expression decreases with increasing stiffness, and one of gene expression recovery at high graft rigidities with |G|>200 Pa). The effect is even more drastic for E-cadherin: protein expression is absent past the bifurcation around |G*|=200 Pa despite strong mRNA expression levels that match those of softer hydrogels, in which there is protein expression of E-cadherin. (FIG. 4). The cells that are directly exposed to external mechanical forces are thus thought able to communicate the signal to adjacent cells at the external surface of the colony.

In this way, by showing that translational control of E-cadherin expression depends on environmental stiffness, signaling mechanisms in hHpSCs with their ability to collectively adapt to the stiffness of their substrate can be linked. Thus, gene-to-protein conversion processes in hHpSCs are subject to stiffness-dependent bifurcation criteria.

Changes in gene expression for hHpSC colonies cultured in KM-HA hydrogels suggest gradual differentiation within these 3D environments. Most notably, differentiation in the present culture model can occur in the absence of biochemical supplementation. These results indicated hHpSCs embedded in various KM-HA hydrogels exhibit differentiation to an intermediate hHB lineage within 1 week of static culture.

Cryopreservation

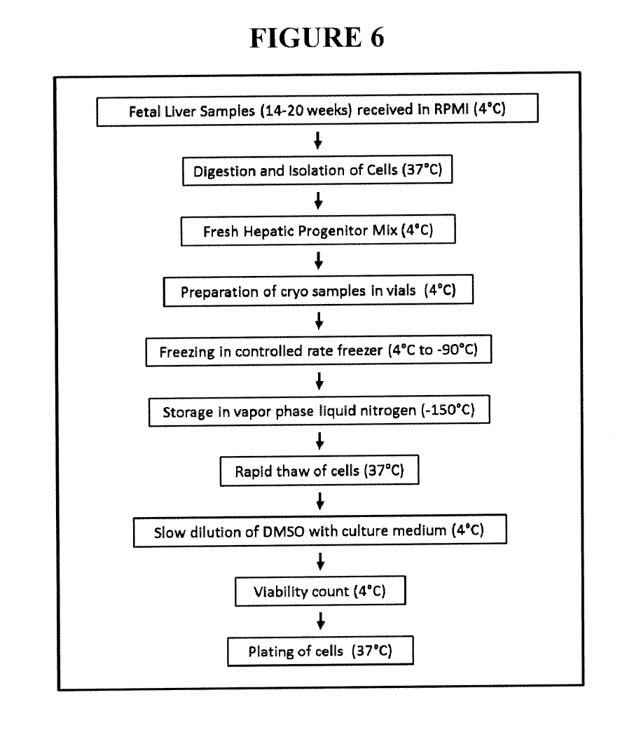

In another embodiment of the present invention HA gels may be used with conventional cryopreservation methods to yield superior preservation and viability upon thawing. An overview of the process is shown in FIG. 6. Without being held to or bound by theory, it is believed that inclusion of HA improves preservation by stimulating adhesion mechanisms (e.g., expression of Integrin β1) that facilitate culturing the cells and preservation of functions post-thawing. Preferably the HA concentration ranges from 0.01 to 1 weight percent, and more preferably from 0.5 to 0.10%.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

The invention now will be described in particularity with the following illustrative examples; however, the scope of the present invention is not intended to be, and shall not be, limited to the exemplified embodiments below.

### Example 1

Mouse hepatic progenitor cells were isolated from a host C57/BL6 mouse (4-5 weeks) according to reported protocols. For the “grafting” studies, a GFP reporter was introduced into the hepatic progenitor cells. The cells were then mixed with hyaluronan (HA) hydrogels and the HA crosslinked with Poly (Ethylene Glycol)-Diacrylate (PEG-DA) prior to introduction into a subject mouse. For introduction/transplantation, mice were anesthetized with ketamine (90-120 mg/kg) and xylazine (10 mg/kg), and their abdomens were opened. The cells, with or without HA, were then slowly injected into the front liver lobe. The incision site was closed and animals were given 0.1 mg/kg buprenorphine every 12 hrs for 48 hrs. After 48 hrs, animals were euthanized, and tissue was removed, fixed, and sectioned for histology.

To determine cell localization within the murine models, “control” hepatic progenitor cells were infected for 4 hrs at 37° C. with a luciferase-expressing adenoviral vector at 50 POI. Survival surgery was performed as described above, and cells (1-1.5E6) were injected directly into the liver lobe with or without HA. Just prior to imaging, mice were injected subcutaneously with luciferin, producing a luminescent signal by the transplanted cells. Using an IVIS Kinetic optical imager, the localization of cells within the mice was determined.

Results

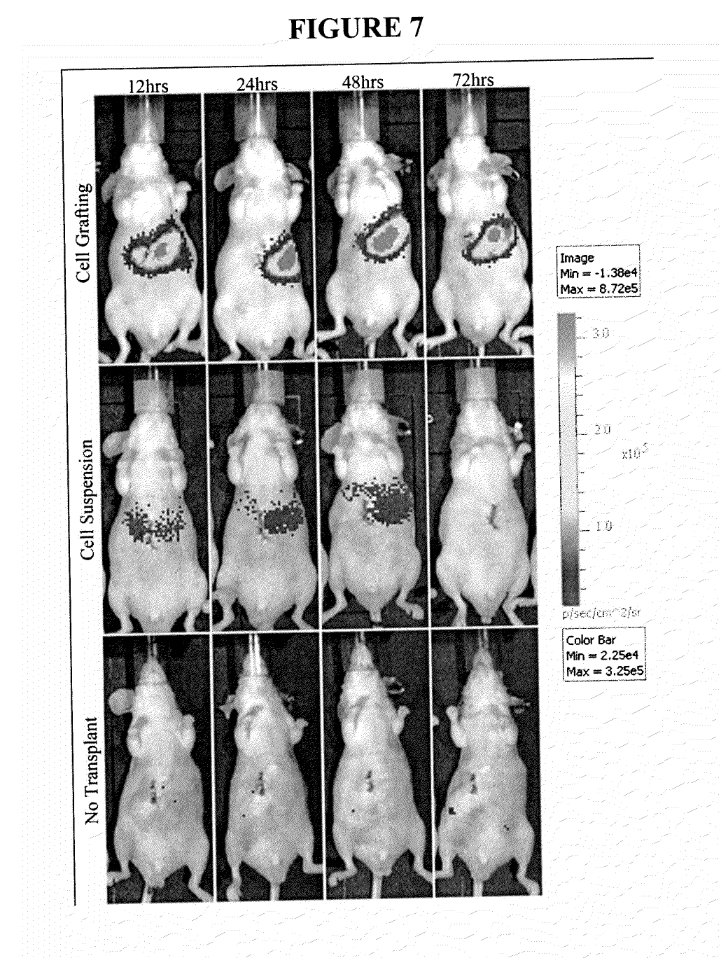

At 24 hrs, “control” cells injected without HA grafting were found both in the liver and lung. At 72 hrs, however, most cells could not be located with only a few identifiable cells remaining in the liver. The grafted cells according to the invention, by contrast, were observed as a group of cells successfully integrated into the liver at both 24 and 72 hrs, and remained present even after two weeks. Cells transplanted via this stem cell niche graft were also seen to localize almost exclusively to liver tissue and were not found in other tissues by assays on randomized histological samples (FIG. 7).

### Example 2

Human hepatic progenitor cells were isolated from fetal liver tissue (16-20 weeks) according to reported protocols. A luciferase-expressing adenoviral vector was introduced into the hepatic progenitor cells. The cells were then mixed with thiol-modified carboxymethyl HA (CMHA-S) and in the presence of the crosslinker Poly (Ethylene Glycol)-Diacrylate (PEG-DA) prior to introduction into a subject mouse. More specifically, the hydrogel was constructed by dissolving HA dry reagents in KM to give a 2.0% solution (weight/volume) and the crosslinker was dissolved in KM to give a 4.0% weight/volume solution. Samples were then allowed to incubate in a 37° C. water bath to completely dissolve. Collagen III and laminin were prepared at a concentration of 1.0 mg/ml and blended with crosslinker/hydrogels in a 1:4 ratio.

For introduction/transplantation, mice were anesthetized with ketamine (90-120 mg/kg) and xylazine (10 mg/kg), and their abdomens were opened. The cells, with or without HA, were then slowly injected into the front liver lobe. The incision site was closed and animals were given 0.1 mg/kg buprenorphine every 12 hrs for 48 hrs. For liver injury models, a one-time dose of carbon tetrachloride (CCl₄) was administered IP at 0.6 ul/g. After 48 hrs, animals were euthanized, and tissue was removed, fixed, and sectioned for histology.

To determine cell localization within the murine models, “control” hepatic progenitor cells were infected for 4 hrs at 37° C. with a luciferase-expressing adenoviral vector at 50 POI. Survival surgery was performed as described above, and cells (1-1.5E6) were injected directly into the liver lobe with or without HA. Just prior to imaging, mice were injected IP with luciferin K salt (150 mg/kg), producing a luminescent signal by the transplanted cells. Using an IVIS Kinetic optical imager, the localization of cells within the mice was determined 10-15 minutes thereafter. (FIG. 7).

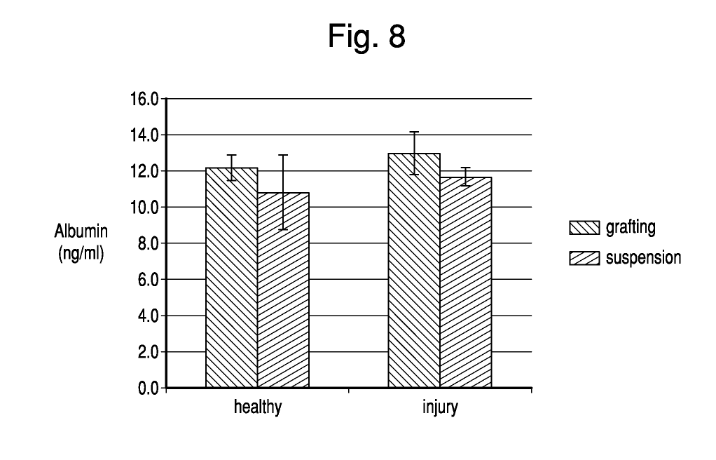

Concentration levels of secreted human albumin in mouse serum at day 7 was assessed to determine the function of the transplanted human hepatic progenitor cells. Albumin production was measured by ELISA with horseradish peroxidase-conjugated fluoroprobes and colorimetric absorbance at 450 nm. (FIG. 8). At day 7, tissue samples were removed from mice and fixed 2 days in 4% PFA and stored in 70% ethanol. 5 μm thick sections were stained for histological examination.

Results

At day 7, blood samples were taken and tissues were removed and fixed for histology. A slight increase in serum albumin was observed in the injury model versus healthy model, and the HA-grafting methods also showed an increase when compared to the results from cell suspensions lacking HA (FIG. 8).

Tissue from the CCl₄-treated mice were stained for human albumin. Cells transplanted via grafting methods using HA were found grouped and maintained large cell masses of transplanted cells within the host cells. Cells transplanted via cell suspension, however, resulted in small aggregates dispersed throughout the liver.

### Example 3

Human pancreatic progenitor cells are isolated from pancreatic tissue. A luciferase-expressing adenoviral vector is introduced into the progenitor cells. The cells are then mixed with thiol-modified carboxymethyl HA (CMHA-S) and in the presence of the crosslinker Poly (Ethylene Glycol)-Diacrylate (PEG-DA) as described in Example 2.

For introduction/transplantation, mice are anesthetized with ketamine (90-120 mg/kg) and xylazine (10 mg/kg), and their abdomens are opened. The cells, with or without HA, are then slowly injected into the pancreas. The incision site is closed and animals are given 0.1 mg/kg buprenorphine every 12 hrs for 48 hrs. After 48 hrs, animals are euthanized, and tissue is removed, fixed, and sectioned for histology.

To determine cell localization within the murine models, “control” progenitor cells are infected for 4 hrs at 37° C. with a luciferase-expressing adenoviral vector at 50 POI. Survival surgery is performed as described above, and cells (1-1.5E6) are injected directly into the pancreas with or without HA. Just prior to imaging, mice are injected IP with luciferin K salt (150 mg/kg), producing a luminescent signal by the transplanted cells. Using an IVIS Kinetic optical imager, the localization of cells within the mice is determined 10-15 minutes thereafter.

Results

At 24 hrs, “control” cells injected without HA grafting are found in the pancreas among other organs. At 72 hrs, however, most cells can not be located with only a few identifiable cells remaining in the pancreas. The grafted cells according to the invention, by contrast, are observed as a group of cells successfully integrated into the pancreas at both 24 and 72 hrs, and remain present even after two weeks.

### Example 4

Studies were performed to assess the viability and function of hepatic stem cells seeded in hydrogels. Viability was assessed in cultures using Molecular Probes Calcein AM live cell viability kit (Molecular Probes, Eugene Oreg.). Membrane-permeant calcein AM was cleaved by esterases in live cells to yield cytoplasmic green fluorescence. Concentration levels of secreted, albumin, transferrin, and urea in culture media were measured during 1 week of culture. Briefly, media supernatant was collected and stored frozen at −20° C. until analyzed. Albumin production was measured by ELISA using human albumin ELISA quantitation sets. Urea production was analyzed using blood urea nitrogen colorimetric reagents. All assays were measured individually with a cytofluor Spectramax 250 multi-well plate reader.

Results

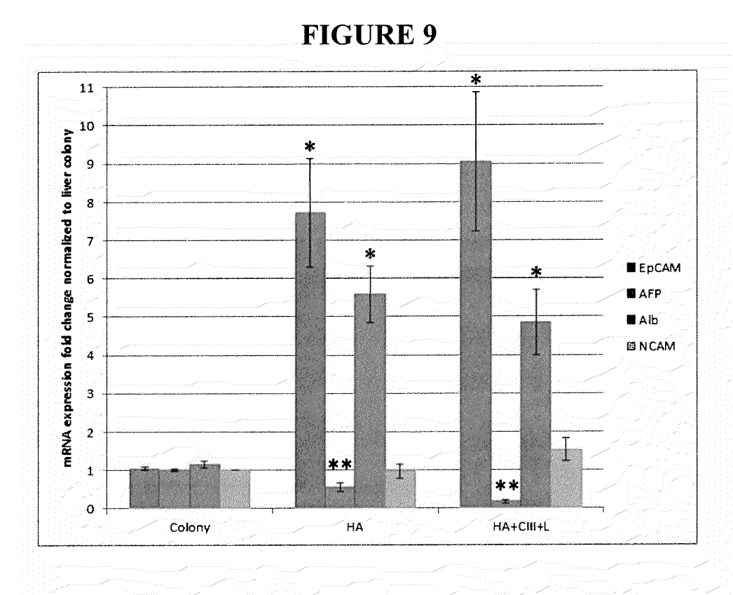

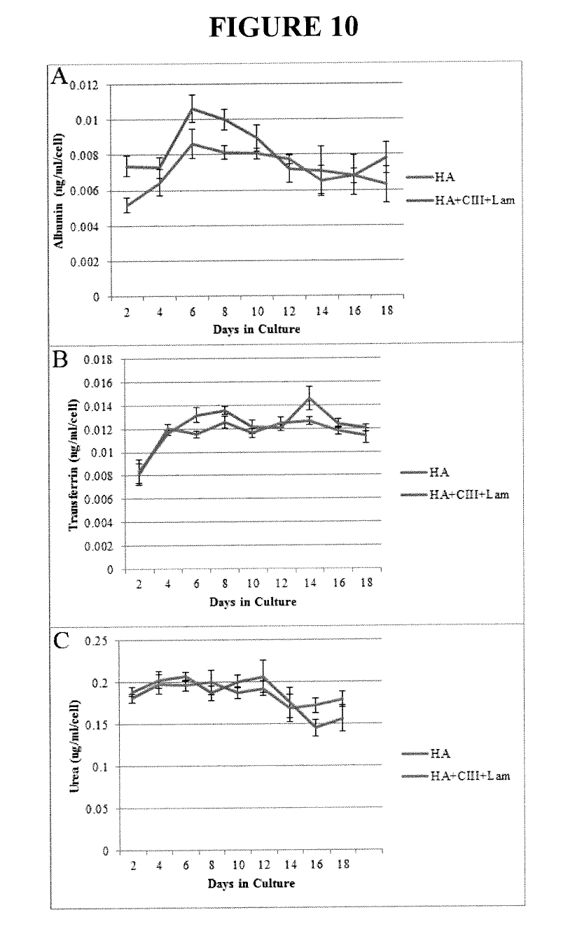

The results are provided in FIGS. 9 and 10. After 3 weeks of culture, cells were analyzed for genetic expression. Levels of mRNA expression were normalized to GAPDH. All measurements are expressed as fold changes compared to initial hepatic stem cell colonies prior to three-dimensional culture in hyaluronan hydrogels. In both experimental hyaluronan culture conditions (HA and HA+collagen III+laminin), there is a significant increase in EpCAM (7.72±1.42, 9.04±1.82) and Albumin (5.57±0.73, 4.84±0.84) when compared to initial colony expression. There was also a significant decrease in the hepatoblast differentiating marker AFP in both conditions (0.55±0.11, 0.17±0.03). In addition, the HA+CIII+Lam condition showed a significant decrease in AFP expression when compared to the basic HA culture.

### Example 5

The effects of mechanical properties of HA hydrogels with diverse concentrations of HA and PEGDA on embedded hHpSCs cultured in a serum-free medium were assessed. The formulations used are summarized in Table 3 below:

| Final contents | | PEGDA initial solution content (1 part) | | | |
|---|---|---|---|---|---|
| (4:1 apportionment) | | 2.0% (w/v) | 4.0% (w/v) | 6.0% (w/v) | 8.0% (w/v) |
| CMHA-S | 1.0% | Formulation A | CMHA-S 0.8% (w/v) | CMHA-S 0.8% (w/v) | Formulation B |
| initial | (w/v) | CMHA-S 0.8% (w/v) | PEGDA 0.8% (w/v) | PEGDA 1.2% (w/v) | CMHA-S 0.8% (w/v) |
| solution | | PEGDA 0.4% (w/v) | | | PEGDA 1.6% (w/v) |
| content | 1.5% | CMHA-S 1.2% (w/v) | Formulation C | Formulation D | CMHA-S 1.2% (w/v) |
| (4 parts) | (w/v) | PEGDA 0.4% (w/v) | CMHA-S 1.2% (w/v) | CMHA-S 1.2% (w/v) | PEGDA 1.6 % (w/v) |
| | | | PEGDA 0.8% (w/v) | PEGDA 1.2% (w/v) | |
| | 2.0 % | Formulation E | CMHA-S 1.6% (w/v) | CMHA-S 1.6% (w/v) | Formulation F |
| | (w/v) | CMHA-S 1.6% (w/v) | PEGDA 0.8% (w/v) | PEGDA 1.2% (w/v) | CMHA-S 1.6% (w/v) |
| | | PEGDA 0.4% (w/v) | | | PEGDA 1.6% (w/v) |

Final KM-HA hydrogel composition for each formulation was achieved by mixing the thiol-modified carboxymethyl HA (CMHA-S) and poly(ethylene glycol)-bis-acrylate (PEGDA) solutions at a 4:1 ratio. Specific concentrations of CMHA-S and PEGDA dry reagents were mixed separately in KM at pH 7.4 at a specific concentration of CMHA-S and of PEGDA, and were warmed for 30 minutes at 37° C. to enhance dissolution of dry reagents. Maximum hydrogel cross-linking occurred without additional media for 1 hour under sterile conditions in an incubator at 5% CO2/air mix and 37° C. Afterwards, hydrogels were supplied with 2.5 ml of HK media and incubated overnight prior to testing.

For diffusivity assays, hydrogel formulations were homogenized by vortexing and plated at ˜1 mm thickness. The hydrogels were incubated without additional media for 1 hour under sterile conditions in an incubator at 5% CO₂/air mix and 37° C. to allow maximum cross-linking after mixing. Samples were then supplemented with equal volumes of additional KM supplied with 2.5 mg/ml (0.036 mM) fluorescein-conjugated 70-kDa Dextran molecules, allowed to diffuse into samples during overnight incubation prior to testing.

Diffusion coefficients of HA hydrogels were measured using a fluorescence recovery after photobleaching (FRAP) system. “In-well” testing was performed on samples after equilibration to room temperature for imaging purposes without prior aspiration of D70-supplemented KM. A total of 5 individual 30-second photobleaching spots (13.5-mW 458/488 nm excitation Argon laser, bleached geometry: 35-um diameter circle) were tested per sample, and a single unidirectional scan pre-bleaching image, a single unidirectional scan image immediately after the end of photobleaching, and 28 unidirectional scan time-series images at 4.0-second delay intervals afterwards (256×256 pixels frame size, 0.9 um/pixel resolution) were acquired for post-processing through a single channel (LP 505 nm, green emission channel).

Results

Stiffness, viscoelastic properties and viscosity of KM-HA hydrogels depend on CMHA-S and PEGDA content. KM-HA hydrogels maintained a constant stiffness across a broad forcing frequency range while exhibiting a perfectly elastic behavior and exhibited shear thinning, as their viscosity decreased with increasing forcing frequency. The content of CMHA-S and PEGDA controlled the mechanical properties of KM-HA hydrogels (FIG. 11a). In contrast, the diffusion properties of KM-HA hydrogels are optimal because they are comparable to that of Kubota's medium alone (FIG. 11b).

Hepatic stem cell colonies were mixed with KM-HA hydrogels and began to abandon their flat configurations in favor of agglomeration to spheroid-like structures or folding into complex 3D structures, both signs of differentiation. After 1 week of culture, cell morphology became diverse and some cells enlarged to about 15 um in size, which is characteristic of hHBs. Immunostaining with antibodies for cell surface markers for hHpSCs and hHBs like EpCAM, CD44 and CDH1 confirmed differentiation.

Throughout culture, hHpSCs in all tested compositions for KM-HA hydrogels secreted AFP and albumin at increasing concentrations, while urea synthesis equilibrated to comparable levels in all KM-HA hydrogels by day 7 (FIG. 12). After 1 week of culture, levels of mRNA expression of EpCAM in hHpSC colony cells seeded within KM-HA hydrogels were significantly higher than those of 2D-grown hHpSC colonies or freshly isolated hHBs. Levels of mRNA expression of NCAM, AFP and E-cadherin (CDH1) for hHpSCs in KM-HA hydrogels were also significantly different from those of 2D-grown hHpSC colonies. (FIG. 5).

Quantitative measurements of gene expression of differentiation markers for hHpSCs (NCAM, AFP, CDH1) and markers common to hHpSCs and hHBs (CD44, EpCAM) exhibit a gradual decrease with increasing KM-HA hydrogel stiffness for |G*|<200 Pa and recovery afterwards (FIG. 5). The cells from all hydrogel formulations expressed EpCAM, NCAM and CD44 protein; however, CD44 appeared enriched in KM-HA formulations with 1.2% CMHA-S or less, whereas NCAM remained rich in all KM-HA hydrogels. (FIG. 4).

### Example 6

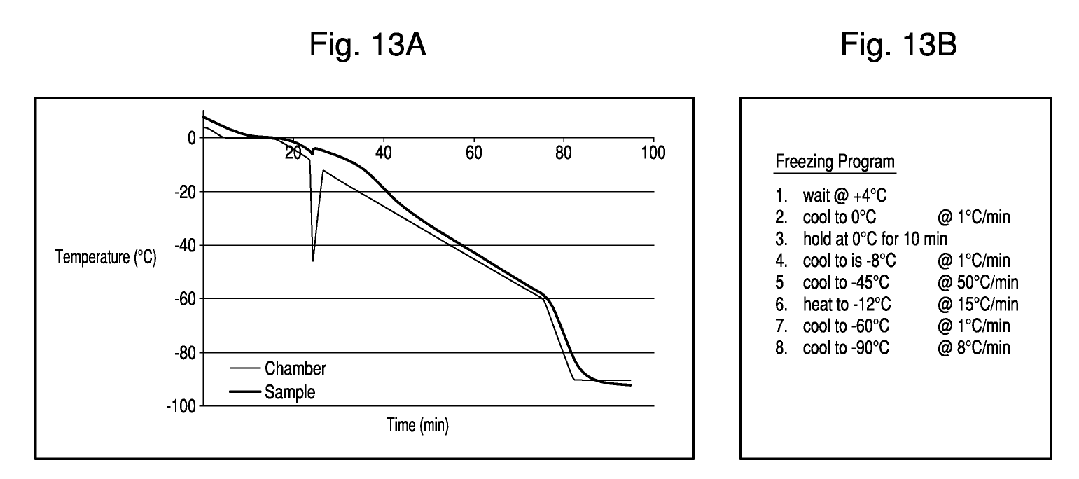

The effects of HA to improve preservation of adhesion mechanisms that could facilitate culturing the cells and preservation of functions post-thawing was assessed. Freshly isolated hHpSCs and hepatoblasts were isolated from fetal livers and cryopreserved in one of a number of different cryopreservation buffers, with or without supplementation of 0.5 or 0.10% hyaluronans (HA). More specifically, samples were frozen at 2×10⁶ cells/ml in cryopreservation solution comprising either culture medium supplemented with 10% DMSO or CryoStor™-CS10 (Biolife Solutions), and with 0, 0.05, or 0.10% HA hydrogel by weight. The cells were allowed to equilibrate in the cryopreservation solution for 10 min at 4° C., before being frozen in uncrosslinked HA in a controlled manner as shown in FIG. 13.

Upon thawing, the cells were plated onto tissue culture plates coated with collagen III at 1 ug/cm² to facilitate stem cell attachment.

Results

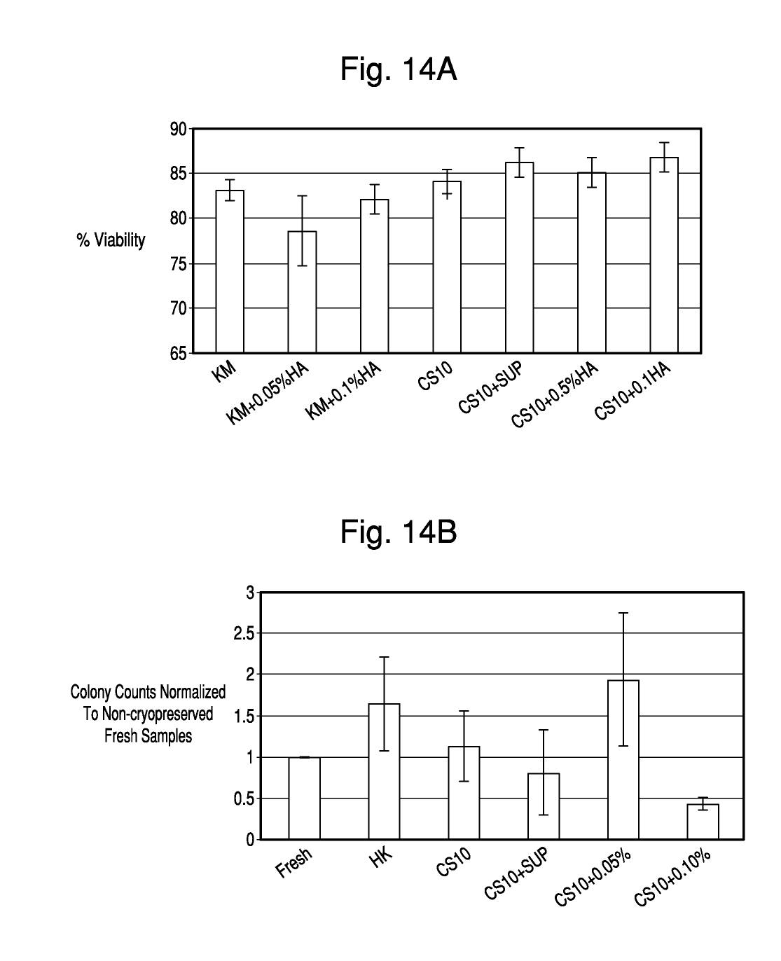

All of the buffers tested yielded high viabilities (80-90%) on thawing (FIG. 14). However, supplementation with HA showed considerable improvement in the ability of the preserved cells to attach to tissue culture surface(s) and to be cultured. Best results observed were for cells cryopreserved in CS10 isotonic medium supplemented with small amounts of hyaluronans (0.05 or 0.10%). The findings reveal improved methods in cryopreservation of freshly isolated human hepatic progenitors under serum-free conditions, offering more efficient methods for stem cell banking in both research and potential therapy applications.

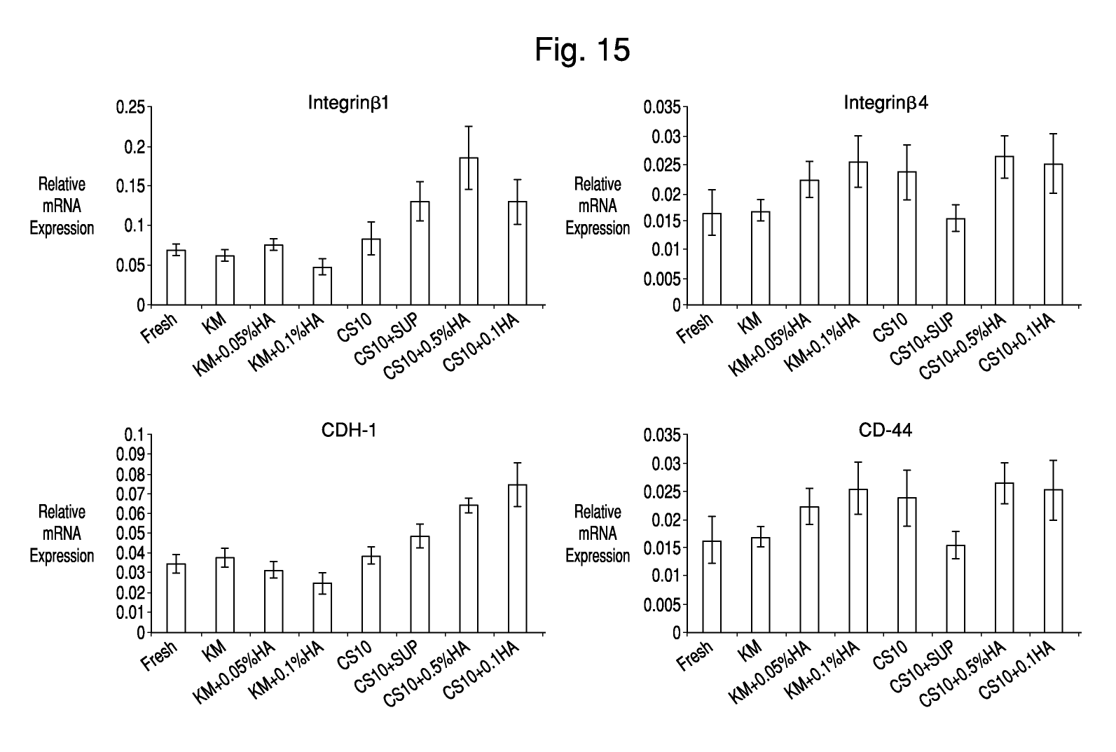

The expression of cell-cell and cell-matrix adhesion factors was determined. A summary of the genetic expression profiles of cell adhesion molecules in cryopreserved samples can be seen in FIG. 15. The highest expression of Integrinβ1 was seen in samples frozen in CS10+0.05% HA (0.130±0.028, n=28). This is significantly different when compared to expression seen in fresh samples (0.069±0.007, n=24, p<0.01). As, well, CDH-1 (E-cadherin) expression in cells frozen in CS10+0.1% HA (0.049±0.006, n=20) and CS10+0.05% HA (0.064±0.003, n=16) showed significant increases in expression when compared to fresh samples (0.037±0.005, n=36, p<0.05).

While the invention has been described in connection with specific embodiments thereof, it will be understood that it is capable of further modifications and this application is intended to cover any variations, uses, or alterations of the invention following. In general, the principles of the invention and including such departures from the present disclosure as come within known or customary practice within the art to which the invention pertains and as may be applied to the essential features hereinbefore set forth and as follows in the scope of the appended claims.

We claim:

1. A method of engrafting cells of an internal organ in a subject having the internal organ in diseased or dysfunctional condition comprising:
a. obtaining normal cells of an internal organ from a donor;
b. combining the cells with one or more gel-forming biomaterials to form a mixture;
c. optionally, combining the mixture with nutrient medium, signaling molecules, extracellular matrix proteins, or a combination thereof;
d. introducing the mixture of step (b) into the subject,
wherein a substantial portion of the cells introduced in step (d) takes up residence in or on at least a portion of the internal organ in vivo.

2. The method according to claim 1, in which the normal cells are stem cells, committed progenitors, or mature cells.

3. The method according to claim 1, in which the internal organ is liver, lung, gut, intestine, heart, kidney, biliary tree, thyroid, thymus, thyroid, brain, or pancreas.

4. The method according to claim 3, in which the internal organ is liver.

5. The method according to claim 3, in which the suspension of cells are liver stem cells, hepatoblasts, committed progenitors of biliary epithelia or hepatocytes, or mature hepatocytes or cholangiocytes.

6. The method according to claim 1, in which the donor is the subject having the internal organ in diseased or dysfunctional condition and the normal cells are obtained from a portion of the internal organ that is not diseased or dysfunctional.

7. The method according to claim 1, in which the donor is a non-autologous donor.

8. The method according to claim 1, in which the donor is a fetus, a neonate, a child, or an adult.

9. The method according to claim 1, in which the additional cells comprise angioblasts, endothelial cells, stellate cell precursors, stellate cells, stromal cells, epithelial stem cells, mature parenchymal cells or combinations thereof.

10. The method according to claim 1, in which the one or more biomaterials comprise collagens, laminins, adhesion molecules, proteoglycans, hyaluronans, glycosaminoglycan chains, chitosan, alginate, and synthetic, biodegradable and biocompatible polymers, or combinations thereof.

11. The method according to claim 1, in which the signaling molecules comprise fibroblast growth factor, hepatocyte growth factor, epidermal growth factor, vascular endothelial cell growth factor (VEGF), insulin like growth factor I, insulin-like growth factor II (IGF-II), oncostatin-M, leukemia inhibitory factor (LIF), interleukins, transforming growth factor-β (TGF-β), HGF, transferrin, insulin, transferrin/fe, tri-iodothyronine, T3, glucagon, glucocorticoids, growth hormones, estrogens, androgens, thyroid hormones, and combinations thereof.

12. The method according to claim 11, in which the interleukins are/IL-6, IL-11, IL-13, or combinations thereof.

13. The method according to claim 1, in which the cells are cultured in serum-free medium.

14. The method according to claim 13, in which the medium comprises insulin, transferrin, lipids, calcium, zinc and selenium.

15. The method according to claim 1, in which the suspension of cells of the internal organ are solidified ex vivo within the biomaterials prior to introducing the cells into the subject.

16. The method according to claim 1, in which the suspension of cells is introduced to or in the proximity of the diseased or dysfunctional tissue.

17. The method according to claim 16, in which the suspension of cells is introduced directly into a tissue.

18. The method according to claim 17, in which the tissue is omentum or liver.

19. The method according to claim 1, in which the suspension of cells is introduced via injection, biodegradable covering, or sponge.

20. A method of repairing tissue of an internal organ in a subject, which internal organ is in a diseased or dysfunctional condition, comprising:
a. obtaining a suspension of normal cells of an internal organ from a donor;
b. combining the cell suspension with one or more biomaterials;
c. optionally combining the cell suspension with growth factors, cytokines, additional cells, or combinations thereof; and
d. introducing the suspension of step (b) or (c) into the subject,
wherein a substantial portion of the cells introduced in step (d) takes up residence in or on at least a portion of the internal organ in vivo.

21. A method of cryopreserving cells, comprising:
a. obtaining cells to be transplanted;
b. combining the cells with gel-forming biomaterials to form a mixture;
c. optionally combining the mixture with one or more of isotonic nutrient medium, signaling molecules and extracellular matrix components;
d. freezing the mixture.

22. The method according to claim 1, in which the one or more biomaterials comprise collagens, laminins, adhesion molecules, proteoglycans, hyaluronans, glycosaminoglycan chains, chitosan, alginate, and synthetic, biodegradable and biocompatible polymers, or combinations thereof.

23. The method according to claim 22, in which the biomaterial comprises hyaluronans.

24. The method according to claim 21, in which the cell suspension is further combined with a cryoprotectant selected from the group consisting of dimethyl sulfoxide(DMSO), glycerol, ethelyene glycol, ethanediol, 1,2-propanediol, 2,-3 butenediol, formamide, N-methylformamide, 3-methoxy-1,2-propanediol by themselves, and combinations thereof.

25. The method according to claim 21, in which the cell suspension is further combined with a sugar, glycine, alanine, polyvinylpyrrolidone, pyruvate, an apoptosis inhibitor, calcium, lactobionate, raffinose, dexamethasone, reduced sodium ions, choline, antioxidants, hormones, or combination thereof.

26. The method according to claim 25, in which the sugar is trehalose, fructose, glucose, or a combination thereof.

27. The method according to claim 25, in which the antioxidants are vitamin E, vitamin A, beta-carotene, or a combination thereof.

28. A tissue graft comprising cells admixed with one or more biomaterials to form a graft, wherein the components of the graft have a shear moduli ranging from 25 to 520 Pa.

29. A method of localizing cells of an internal organ onto a surface, into an interior portion, or both of a target internal organ comprising introducing a preparation comprising cells of an internal organ and a solution of one or more hydrogel-forming precursors, in the presence of an effective amount of a cross-linker, onto a surface, into an interior portion, or both of a target internal organ in vivo, which preparation forms a hydrogel comprising cells of an internal organ on a surface, in an interior portion, or both of a target internal organ.

30. The method of claim 29 in which cells of an internal organ are localized for a period of at least twelve hours, at least twenty-four hours, or at least about forty-eight hours or at least seventy-two hours, onto a surface, into an interior portion, or both of a target internal organ.

31. The method of claim 29 in which the cells of an internal organ are not tumor or cancer cells or diseased cells.

32. The method of claim 29 in which the cells of an internal organ are normal cells, tumor or cancer cells, or cells infected with a pathogen selected from the group consisting of virus, bacteria, malaria.

33. The method of claim 29 in which the one or more hydrogel-forming precursors comprise glycosaminoglycans, hyaluronans, proteoglycan, gelatins, collagens, laminins, other attachment proteins, plant-derived matrix components, denatured forms thereof, or combinations thereof.

34. The method of claim 29 in which the one or more hydrogel-forming precursors are comprised of a thiol-modified sodium hyaluronate and a thiol-modified gelatin.

35. The method of claim 29 in which the cross-linker comprises polyethylene glycol diacrylate or a disulfide-containing derivative thereof.

36. The method of claim 29 in which the hydrogel possesses a viscosity ranging from about 0.1 to about 100 kPa, preferably about 1 to about 10 kPa, more preferably about 2 to about 4 kPa.

37. The method of claim 29 in which the preparation, in the presence of an effective amount of a cross-linker, is introduced into a pocket formed on a surface of a target internal organ.

38. The method of claim 37 in which the pocket if prepared from omentum, spider silk, and/or insect silk.

39. The method of claim 29 in which the target internal organ is selected from the group consisting of liver, pancreas, biliary tree, thyroid, intestine, lung, prostate, breast, brain, uterus, bone, or kidney.

40. The method of claim 29 in which the hydrogel forms in situ.
